(19) United States
(12) Patent Application Publication
(10) Pub. No.: US 2025/0275981 A1
(43) Pub. Date: Sep. 4, 2025

(54) HOMOHARRINGTONINE AND PACLITAXEL COMBINATION FOR THE TREATMENT OF BREAST CANCER

(71) Applicant: University of Saskatchewan, Saskatoon (CA)

(72) Inventors: Deborah Helen Anderson, Saskatoon (CA); Riley Plett, Saskatoon (CA); Paul Mellor, Saskatoon (CA); Franco Vizeacoumar, Saskatoon (CA)

(73) Assignee: University of Saskatchewan, Saskatoon, SK (CA)

(21) Appl. No.: 18/857,957

(22) PCT Filed: Apr. 19, 2023
(86) PCT No.: PCT/CA2023/050529

§ 371 Date: Oct. 18, 2024

Related U.S. Application Data
(60) Provisional application No. 63/333,326, filed on Apr. 21, 2022.

(51) Int. Cl. A61K 31/55; A61K 31/337; A61K 45/06; A61P 35/00

(52) U.S. Cl. CPC A61K 31/55; A61K 31/337; A61K 45/06; A61P 35/00

(57) ABSTRACT

The present application relates to combination treatments for breast cancer. For example, the application relates to the use of homoharringtonine and paclitaxel for the treatment of breast cancer including, for example, triple negative breast cancer.

### RELATED APPLICATIONS

The present application claims the benefit of priority of co-pending U.S. provisional patent application No. 63/333,326 filed on Apr. 21, 2022 the contents of which are incorporated herein by reference in their entirety.

### FIELD

The present application relates to combination therapies for the treatment of breast cancer. For example, the present application relates to the use homoharringtonine and paclitaxel for treatment of breast cancer including triple negative breast cancer.

### BACKGROUND

Breast cancer is the most common cancer in Canadian women, with a prevalence of 26% or ˜25,000 new cases each year [1]. Women with metastatic breast cancer have a disheartening 5-year survival rate of only 28% [2]. There are three main subtypes of breast cancer categorized by expression of their estrogen receptor (ER), progesterone receptor (PR) and human epidermal growth factor receptor (HER2) [3]. Luminal breast cancers (65-75%) express estrogen and/or progesterone receptors and are typically treated with anti-estrogens [4]. HER2+ breast cancers (1015%) overexpress the HER2 receptor and are treated with antibodies to HER2 such as trastuzumab [4]. Triple negative breast cancers (TNBCs; 15-20%) lack the three receptors that define the other subtypes. Since they are missing these molecular targets, TNBCs are treated with various combinations of general cytotoxic chemotherapy agents as the primary treatment [5]. Thus, there is an unmet clinical need to develop treatments for TNBC that are both effective and have fewer toxic side-effects.

CREB3 (CAMP responsive element binding protein 3)-family transcription factors have important roles in tissue development, lipid metabolism, protein secretion and tumorigenesis [6]. Their normal roles are to provide cell-type specific functions to different tissues to maintain cellular homeostasis in response to cell stress by regulating cell secretory capacity and cell specific cargos [7-9]. They are endoplasmic reticulum transmembrane proteins and in response to cell stress they traffic to the Golgi complex to be cleaved by Golgi-resident S1P and S2P proteases to generate active cytosolic transcription factors that enter the nucleus to regulate gene expression [6].

It has been shown that a member of CREB3-family, CREB3L1 (CAMP responsive element binding protein 3 like 1), is a transcription factor responsible for repressing the expression of genes that promote breast cancer progression and metastasis [10, 11]. CREB3L1 expression is frequently upregulated in early breast cancers, but its expression is significantly reduced in more advanced and metastatic breast cancers due to epigenetic silencing [11, 12]. Loss of CREB3L1 expression is associated with poor prognosis and reduced patient survival times in luminal A (ER+, HER2−) and TNBCs [11, 13].

Overall, approximately 30% of breast cancers lack CREB3L1 expression [11, 14]. CREB3L1-deficient breast cancers are typically the more advanced metastatic breast tumors and include approximately 75% of TNBCs [14]. It has been shown that CREB3L1 loss directly contributes to the metastatic phenotype of breast cancer cells, using cell-based assays and animal models of breast cancer [10, 11, 14]. Re-expression of CREB3L1 in CREB3L1-deficient breast cancer cells significantly decreases metastatic properties (increased growth in soft agar, migration, invasion) [10, 14]. Consistent with these results, poorly metastatic CREB3L1-expressing cells can be converted to more metastatic phenotypes by CREB3L1 knockdown [10].

Tumor formation and metastasis have also been characterized using a syngeneic rat mammary tumor model [10]. CREB3L1-deficient cells formed tumors at high frequencies, and most had lymph node metastases. The same cells engineered to stably express CREB3L1 formed primary tumors at a reduced frequency at day 30 and by day 60 many of these regressed to a nearly undetectable size and none formed metastases [10]. Blood vessel formation within the regressing CREB3L1-expressing tumors was significantly decreased compared to tumors from CREB3L1-deficient animals (p<0.001) [10]. These results suggest that CREB3L1 blocks angiogenesis, which is necessary for large tumors to survive and plays a role in metastasis suppression. Additionally, re-expression of CREB3L1 in a mouse xenograft model of human TNBC similarly showed reduced tumor progression and lung metastases, as compared to the CREB3L1-deficient parental TNBC, further supporting the role of CREB3L1 as a metastasis suppressor [14].

### SUMMARY

The Applicant has conducted large scale screen of FDA-approved drugs to identify effective new drug(s) for the treatment of breast cancer, such as CREB3L1-deficient metastatic triple negative breast cancer (TNBCs) and luminal A breast cancer. After further screening and testing, four FDA-approved drugs were identified and were then separately evaluated for possible synergy with doxorubicin and paclitaxel in TNBC cells lines. A strong synergy was exhibited by homoharringtonine in combination with paclitaxel. The synergy was observed at doses that were considerably lower than the doses (for example, EC₅₀ values) of the individual drugs when used alone, and neither drug was found to show such a strong synergy with any other tested drug. Therefore, the Applicants have found that the combination of homoharringtonine and paclitaxel is very effective in killing breast cancer cells such as TNBC using concentrations of each drug that are lower that are presently used in treatments where these compounds are used alone. Accordingly, in some embodiments, the present application includes the combination of homoharringtonine and paclitaxel as an effective treatment for breast cancer such as TNBC. In some embodiments, concentrations of each drug are used that are lower that are presently used in treatments when using each drug alone. Accordingly, concentrations of each of these compounds are administered or used such that any toxic side effects are minimized.

Accordingly, the present application includes a method of treating breast cancer comprising administering to a subject in need thereof, an effective amount of paclitaxel, or a pharmaceutically acceptable salt, prodrug and/or solvate thereof, in combination with an effective amount of homoharringtonine or a pharmaceutically acceptable salt, prodrug and/or solvate thereof.

The present application also includes a method of improving the efficacy of homoharringtonine for treating breast cancer comprising administering, to a subject in need thereof, an effective amount of paclitaxel, or a pharmaceutically acceptable salt, prodrug and/or solvate thereof, in combination with an effective amount of homoharringtonine or a pharmaceutically acceptable salt, prodrug and/or solvate thereof.

The present application also includes a method of preventing or reducing breast cancer metastasis comprising administering, to a subject in need thereof, an effective amount of homoharringtonine, or a pharmaceutically acceptable salt, prodrug and/or solvate thereof, in combination with an effective amount of paclitaxel or a pharmaceutically acceptable salt, prodrug and/or solvate thereof.

In some embodiments, the breast cancer is triple negative breast cancer.

The present application also includes a pharmaceutical composition comprising homoharringtonine or a pharmaceutically acceptable salt, prodrug and/or solvate thereof and paclitaxel or a pharmaceutically acceptable salt, prodrug and/or solvate thereof wherein the homoharringtonine and paclitaxel are present in amounts that are effective to treat breast cancer, for improving the efficacy of homoharringtonine for treating breast cancer or for preventing or reducing breast cancer metastasis.

The present application also includes a kit for the treatment breast cancer, the kit comprising paclitaxel or a pharmaceutically acceptable salt, prodrug and/or solvate thereof and homoharringtonine or a pharmaceutically acceptable salt, prodrug and/or solvate thereof and optionally instructions for administration of paclitaxel or a pharmaceutically acceptable salt, prodrug and/or solvate thereof and homoharringtonine, or a pharmaceutically acceptable salt and/or solvate thereof, to a subject in need thereof.

The present application also includes a kit for the treatment of a breast cancer, the kit comprising: homoharringtonine or a pharmaceutically acceptable salt and/or solvate thereof; and instructions for administration of homoharringtonine, or a pharmaceutically acceptable salt and/or solvate thereof, to a subject being administered paclitaxel for treating breast cancer.

The present application also includes a kit for the treatment of a breast cancer, the kit comprising: paclitaxel or a pharmaceutically acceptable salt and/or solvate thereof; and instructions for administration of paclitaxel, or a pharmaceutically acceptable salt and/or solvate thereof, to a s administered homoharringtonine for treating breast cancer.

The present application also includes a kit for improving the efficacy of homoharringtonine for the treatment of breast cancer, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

The present application also includes a kit for improving the efficacy of homoharringtonine for the treatment breast cancer, the kit comprising homoharringtonine, or a pharmaceutically acceptable salt and/or solvate thereof; and instructions for administration of homoharringtonine, or a pharmaceutically acceptable salt and/or solvate thereof, to a subject being administered paclitaxel for the treatment of breast cancer.

The present application also includes a kit for preventing or reducing breast cancer metastasis, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 is a diagram showing the primary and secondary screen schematic.

FIG. 2 contains graphs showing drugs that are more cytotoxic towards CREB3L1-deficient HCC1806 cells (black circles), as compared to HCC1806+HA-CREB3L1 cells (black squares). Cells were plated and after 24 hours were treated with the indicated concentration of drug, or solvent control, for 4 days. Solvents (max 0.4%) had little or no effect on the cell growth/number. Cells were stained, imaged and counted. Cell viability (%) was calculated as (# live cells in experimental well)/(# live cells in solvent control well)*100. Mean % viability±SEM from triplicate measurements from at least 3 independent experiments, a) Palbociclib Isethionate, 1:3 serial dilutions for concentrations 0-50 μM. b) Cladribine, 1:3 serial dilutions for concentrations 0-9 μM. c) Lanatoside C, 1:2 serial dilutions for concentrations 0-2 μM. d) Homoharringtonine, 1:3 serial dilutions for concentrations 0-333 nM.

FIG. 3 contains graphs showing various drug cytotoxicities towards non-tumorigenic MCF10A cells (squares) as compared to CREB3L1-deficient HCC1806 cells (circles). Cells were plated and after 24 hours were treated with the indicated concentration of drug, or solvent control, for 4 days. Solvents (max 0.4%) had little or no effect on the cell growth/number. Cells were stained, imaged and counted. Cell viability (%) was calculated as (# live cells in experimental well)/(# live cells in solvent control well)*100. Mean % viability±SEM from triplicate measurements from at least 3 independent experiments, a) Palbociclib Isethionate, 1:3 serial dilutions for concentrations 0-50 μM. b) Cladribine, 1:3 serial dilutions for concentrations 0-1 μM. c) Lanatoside C, 1:2 serial dilutions for concentrations 0-2 μM. d) Homoharringtonine, 1:3 serial dilutions for concentrations 0-333 nM.

FIG. 4 contains graphs showing various drug sensitivities across a panel of CREB3L1-deficient luminal A and TNBC cell lines. Cell viability was measured after 4 days of drug treatment over a range of drug concentrations as before. EC₅₀ values were determined as per Table 1 and plotted here for comparison.

FIG. 5 contains a graph showing paclitaxel sensitivity for HCC1806+HACREB3L1 cells (squares) and CREB3L1-deficient HCC1806 cells (circles). Cells were plated and after 24 hours were treated with the indicated concentration of drug, or solvent control, for 4 days. Solvents (max 0.4%) had little or no effect on the cell growth/number. Cells were stained, imaged and counted. Cell viability (%) was calculated as (# live cells in experimental well)/(# live cells in solvent control well)*100. Mean % viability±SEM from duplicate or triplicate measurements from at least 3 independent experiments. 1:3 serial dilutions for concentrations 0-111 nM.

FIG. 6 contains diagrams showing the assessment of possible drug synergy between palbociclib isethionate and either doxorubicin (a, b, c, d) or paclitaxel (e, f, g, h). Cell viability (a, b, e, f) was measured after 4 days of drug treatment over a range of drug concentrations for HCC1806 (a, c, e, g) and HCC1806+HA-CREB3L1 (b, d, f, h) cells. Palbociclib Isethionate EC₅₀ for HCC1806=500 nM; 1:3 serial dilutions (0-9 μM). Doxorubicin EC₅₀ for HCC1806=13 nM; 1:3 serial dilutions (0-111 nM). Paclitaxel EC₅₀ for HCC1806=1.2 nM; 1:3 serial dilutions (0-111 nM). A zero interaction potency score (ZIP; c, d, g, h) was calculated by the SynergyFinder®-package for each data point to measure possible drug synergy; ZIP >10 indicates synergy.

FIG. 7 contains diagrams showing the assessment of possible drug synergy between cladribine and either doxorubicin (a, b, c, d) or paclitaxel (e, f, g, h). Cell viability (a, b, e, f) was measured after 4 days of drug treatment over a range of drug concentrations for HCC1806 (a, c, e, g) and HCC1806+HA-CREB3L1 (b, d, f, h) cells. Cladribine EC₅₀ for HCC1806=92 nM; 1:3 serial dilutions (0-9 μM). Doxorubicin EC₅₀ for HCC1806=13 nM; 1:3 serial dilutions (0-111 nM). Paclitaxel EC₅₀ for HCC1806=1.2 nM; 1:3 serial dilutions (0-111 nM). A zero interaction potency score (ZIP; c, d, g, h) was calculated by the SynergyFinder®-package for each data point to measure possible drug synergy; ZIP >10 indicates synergy.

FIG. 8 contains diagrams showing the assessment of possible drug synergy between lanatoside C and either doxorubicin (a, b c, d) or paclitaxel (e, f, g, h). Cell viability (a, b, e, f) was measured after 4 days of drug treatment over a range of drug concentrations for HCC1806 (a, c, e, g) and HCC1806+HA-CREB3L1 (b, d, f, h) cells. Lanatoside C EC₅₀ for HCC1806=143 nM; 1:2 serial dilutions (0-2 μM). Doxorubicin EC₅₀ for HCC1806=13 nM; 1:3 serial dilutions (0-111 nM). Paclitaxel EC₅₀ for HCC1806=1.2 nM; 1:3 serial dilutions (0-111 nM). A zero interaction potency score (ZIP; c, d, g, h) was calculated by the SynergyFinder®-package for each data point to measure possible drug synergy; ZIP >10 indicates synergy.

FIG. 9 contains diagrams showing the assessment of possible drug synergy between homoharringtonine and either doxorubicin (a, b, c, d) or paclitaxel (e, f, g, h). Cell viability (a, b, e, f) was measured after 4 days of drug treatment over a range of drug concentrations for HCC1806 (a, c, e, g) and HCC1806+HA-CREB3L1 (b, d, f, h) cells. Homoharringtonine EC₅₀ for HCC1806=29 nM; 1:3 serial dilutions (0-333 nM). Doxorubicin EC₅₀ for HCC1806=13 nM; 1:3 serial dilutions (0-111 nM). Paclitaxel EC₅₀ for HCC1806=1.2 nM; 1:3 serial dilutions (0-111 nM). A zero interaction potency score (ZIP; c, d, g, h) was calculated by the SynergyFinder®-package for each data point to measure possible drug synergy; ZIP >10 indicates synergy.

FIG. 10 contains diagrams showing that homoharringtonine and paclitaxel show synergistic cytotoxicity across several triple negative breast cancer (TNBC) cell lines. Cell viability was measured after 4 days of drug treatment over a range of homoharringtonine (HHT) and paclitaxel (PTX) concentrations for HCC1806 (a), HCC38 (c), HCC1395 (e), and Hs578T (g) TNBC cells. A zero-interaction potency score (ZIP; b, d, f, h) was calculated by the SynergyFinder®-package for each data point to measure possible drug synergy; ZIP >10 indicates synergy. Maximum values are bolded. Cytotoxicity and ZIP synergy scores at HHT (12 nM) and PTX (0.15 nM) are also shown although not bolded unless also the maximum values.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in the present application, the singular forms “a”, “an” and “the” include plural references unless the content clearly dictates otherwise. For example, an embodiment including “a compound” should be understood to present certain aspects with one compound, or two or more additional compounds.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds exist as individual salts and hydrates, as well as a combination of, for example, a solvate of a salt of the compounds.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure reactant ratio and whether or not the reaction should be performed under an anhydrous or inert atmosphere, can be varied to optimize the yield of the desired product and it is within their skill to do so.

The term “homoharringtonine” as used herein refers to a compound having the chemical name: 1-((1S,3aR,14bS)-2-Methoxy-1,5,6,8,9,14b-hexahydro-4H-cyclopenta(a)(1,3)dioxolo(4,5-h)pyrrolo (2,1-b)(3)benzazepin-1-yl) 4-methyl (2R)-2-hydroxy-2-(4-hydroxy-4-methylpentyl)butanedioate cephalotaxine, or 4-methyl (2R)-hydroxyl-2-(4-hydroxyl-4-methylpentyl) butanedioate, also known as “omacetaxine mepesuccinate” and having the chemical Formula:

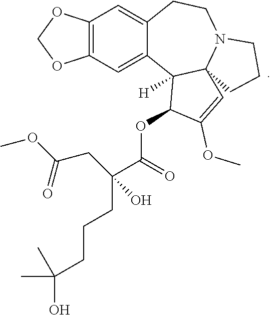 [SMILES: COC(=O)C[C@](O)(CCCC(C)(C)O)C(=O)O[C@@H]1C(OC)=C[C@]23CCCN2CCc2cc4c(cc2[C@H]13)OCO4]

The term “paclitaxel” as used herein refers to a compound having the chemical name: (2α,4α,5β,7β,10β,13α)-4, 10-Bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate and having the chemical formula:

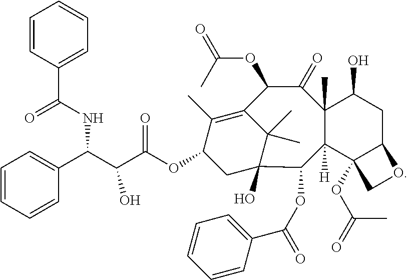 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

The term “pharmaceutically acceptable salt” means an acid addition salt or a basic addition salt suitable for, or compatible with, the treatment of subjects.

The term “pharmaceutically acceptable carrier” means a non-toxic solvent, dispersant, excipient, adjuvant or other material which is mixed with an active ingredient (for example, one or both of paclitaxel and homoharringtonine to permit the formation of a pharmaceutical composition, i.e., a dosage form capable of administration to a subject.

The term “pharmaceutically acceptable” means compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvates” as used herein refers to complexes formed between a compound and a solvent from which the compound is precipitated or in which the compound is made. Accordingly, the term “solvate” as used herein means a compound, or a salt and/or prodrug of a compound, wherein molecules of a suitable solvent are incorporated in the crystal lattice.

The term “prodrug” as used herein means a compound, or salt and/or solvate of a compound, that, after administration, is converted into an active drug.

The term “cell” as used herein refers to a single cell or a plurality of cells and includes a cell either in a cell culture or in a subject.

The term “subject” as used herein includes all members of the animal kingdom including mammals. Thus, the methods and uses of the present application are applicable to both human therapy and veterinary applications.

The term “pharmaceutical composition” as used herein refers to a composition of matter for pharmaceutical use.

The term “parenteral” as used herein means taken into the body or administered in a manner other than through the gastrointestinal tract.

The term “administered” as used herein means administration of an effective amount of a compound or compounds to a cell either in cell culture or in a subject.

The term “synergistically effective amount” refers to the amount of one compound of a drug combination necessary to elicit a synergistic effect with another compound present in the drug combination.

The term “synergistically effective” as used herein means that the therapeutic effect observed following administration of two or more compounds is greater than the sum of the therapeutic effects observed following the administration of each single compound.

As used herein, the term “effective amount” or “therapeutically effective amount” means an amount effective, at dosages and for periods of time necessary to achieve a desired result.

The terms “to treat”, “treating” and “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of a disease, disorder or condition, stabilized (i.e. not worsening) state of a disease, disorder or condition, preventing spread of a disease, disorder or condition, delay or slowing of a disease, disorder or condition progression, amelioration or palliation of a disease, disorder or condition state, diminishment of the reoccurrence of a disease, disorder or condition, inhibiting or reducing a disease, disorder or condition and remission (whether partial or total), whether detectable or undetectable. “Treating” and “treatment” can also mean prolonging survival as compared to expected survival if not receiving treatment. “Treating” and “treatment” as used herein also include prophylactic treatment.

“Palliating” a disease, disorder or condition means that the extent and/or undesirable clinical manifestations of the disease, disorder or condition are lessened and/or time course of the progression is slowed or lengthened, as compared to not treating the disease, disorder or condition.

The term “prevention” or “prophylaxis”, or synonym thereto, as used herein refers to a reduction in the risk or probability of a subject becoming afflicted with breast cancer or manifesting a symptom associated with a breast cancer. In addition or alternatively “prevention” or “prophylaxis” refers a reduction in the risk or probability of a subject becoming afflicted with breast cancer metastasis.

By “preventing or reducing brain cancer metastasis” means reduction and/or slowing of breast cancer metastasis compared to otherwise the same conditions, except for in the absence in the compounds, and includes slowing and/or disruption in the rate of breast cancer metastasis.

The term “administered” as used herein means administration of a therapeutically effective amount of a compound, or one or more compounds, or a composition to a cell or a subject.

The term “triple negative breast cancer” or “TNBC” as used herein refers to breast cancers wherein at least a portion of the cancer cells are negative for the estrogen receptor (ER) protein, negative for the progesterone receptor (PR) protein and negative for the human epidermal growth factor receptor 2 (HER2) protein.

The term “basal-like breast cancer” as used herein refers to a subtype of breast cancer comprising a heterogenous group of tumors characterized by the absence of, or low levels of expression of, estrogen receptors, very low prevalence of Her2 overexpression and expression of genes usually found in the basal or myoepithelial cells of the human breast.

The term “luminal A breast cancer” as used herein refers to breast cancers wherein at least a portion of the cancer cells are ER positive and PR positive, but negative for HER2.

The term “luminal B breast cancer” as used herein refers to breast cancers wherein at least a portion of the cancer cells are ER positive, HER2 amplified, and negative for PR.

The term “HER2+ breast cancer” refers to breast cancers wherein at least a portion of the cancer cells are negative for ER and PR, but amplified for HER2.

The term “hormone receptor positive breast cancer” refers to breast cancers wherein at least a portion of the cancer cells are ER positive and/or PR positive.

The term “hormone receptor negative breast cancer” refers to breast cancers wherein at least a portion of the cancer cells are negative for ER protein and/or PR protein.

The term “estrogen receptor (ER)-positive breast cancer” refers to breast cancers wherein at least a portion of the cancer cells are ER positive.

The term “progesterone receptor (PR)-positive breast cancer” refers to breast cancers wherein at least a portion of the cancer cells are PR positive.

The term “estrogen receptor (ER)-negative breast cancer” refers to breast cancers wherein at least a portion of the cancer cells are negative for ER protein.

The term “progesterone receptor (PR)-negative breast cancer” refers to breast cancers wherein at least a portion of the cancer cells are negative for PR protein.

The term “BRCA1 breast cancer” refers to breast cancers wherein at least a portion of the cancer cells are characterized with a mutation in the BRCA1 gene and/or reduced wild type BRCA1 expression.

The term “BRCA2 breast cancer” refers to breast cancers wherein at least a portion of the cancer cells are characterized with a mutation in the BRCA2 gene and/or reduced wild type BRCA2 expression.

The term “breast cancer” as used herein refers generally to the uncontrolled growth of breast tissue and characterized by anomalous rapid proliferation of abnormal cells in one or both breasts of a subject.

When used, for example, with respect to the methods of treatment, uses, compositions and kits of the application, a subject, for example a subject “in need thereof” is a subject who has been diagnosed with, is suspected of having breast cancer.

The term “CREB3L1” as used herein refers to CREB3-like 1.

The term “CREB3” as used herein refers to CAMP responsive element binding protein 3.

The term “ER” as used herein refers to estrogen receptor.

The term “PR” as used herein refers to progesterone receptor.

The term “HA” as used herein refers to hemagglutinin.

The term “HER2” as used herein refers to human epidermal growth factor receptor 2.

The term “PBS” as used herein refers to phosphate buffered saline.

The term “PR” as used herein refers to progesterone receptor.

The term “RFP” as used herein refers to red fluorescent protein.

The term “ZIP” as used herein refers to Zero Interaction Potency.

### II. Treatment Methods and Uses of the Application

The molecular signature of CREB3L1-deficiency was used in a large scale screen of FDA-approved drugs to identify effective new drug(s) for the treatment of CREB3L1-deficient metastatic triple negative breast cancer (TNBCs) and luminal A breast cancer. In particular, four FDA-approved drugs were identified including the CDK4/6 inhibitor palbociclib isoethionate, the Na+/K+-ATPase inhibitor lanatocide C (also named isolanid), the nucleoside analog cladribine, and the protein translation inhibitor homoharringtonine (also named omacetaxine mepesuccinate), that were found to be preferentially more cytotoxic towards CREB3L1-deficient TNBCs and luminal A breast cancer cells, as compared to the same cells re-expressing CREB3L1.

All four identified drugs were then separately evaluated for possible synergy with two known chemotherapy agents, the anthracycline doxorubicin and the microtubule stabilizing agent paclitaxel, in TNBC cells lines. It was unexpectedly found that a strong synergy was exhibited by homoharringtonine in combination with paclitaxel. Further, the synergy was observed at doses that were considerably lower than the doses (for example, EC₅₀ values) of the individual drugs when used alone, and neither drug was found to show such a strong synergy with any other tested drug. Therefore, the Applicants have found that the combination of homoharringtonine and paclitaxel is very effective in killing breast cancer cells such as TNBC using lower concentrations of each drug. Accordingly, in some embodiments, the present application includes the combination of homoharringtonine and paclitaxel as an effective treatment for breast cancer such as TNBC. In some embodiments, concentrations of each drug are used that are lower that are presently used in treatments when using each drug alone. Accordingly concentrations of each of these compounds are used such that any toxic side effects are minimized.

Accordingly, the present application includes a method of treating breast cancer in a subject comprising administering, to a subject in need thereof, an effective amount of homoharringtonine, or a pharmaceutically acceptable salt, prodrug and/or solvate thereof, in combination with an effective amount of paclitaxel or a pharmaceutically acceptable salt, prodrug and/or solvate thereof.

The present application also includes a use of an effective amount homoharringtonine, or a pharmaceutically acceptable salt, prodrug and/or solvate thereof, in combination with an effective amount of paclitaxel or a pharmaceutically acceptable salt, prodrug and/or solvate thereof, for treating breast cancer in a subject; a use of an effective amount of homoharringtonine, or a pharmaceutically acceptable salt, prodrug and/or solvate thereof, in combination with an effective amount of paclitaxel or a pharmaceutically acceptable salt, prodrug and/or solvate thereof, for preparation of a medicament for treating breast cancer in a subject; and an effective amount of homoharringtonine, or a pharmaceutically acceptable salt, prodrug and/or solvate thereof, in combination with an effective amount of paclitaxel or a pharmaceutically acceptable salt, prodrug and/or solvate thereof, for use to treat breast cancer in a subject.

In some embodiments, treating breast cancer is by inhibiting breast cancer cell proliferation. In some embodiments, inhibiting breast cancer cell proliferation includes delaying or slowing of breast cancer cell growth, inducing breast cancer cell death, reducing breast cancer cell viability, inhibiting or delaying tumor growth, and/or or reducing tumor size.

In the context of the present application, “inhibiting breast cancer cell proliferation” refers to any reduction of breast cancer cell proliferation compared to otherwise the same conditions, except for in the absence in the compounds, and includes any delay or slowing of breast cancer cell growth, induction of breast cancer cell death, reduction breast cancer cell viability, inhibition or delay of tumor growth, and/or or reduction in tumor size compared to otherwise the same conditions, except for in the absence in the compounds.

In some embodiments, the present application includes a method of improving the efficacy of homoharringtonine for treating breast cancer comprising administering, to a subject in need thereof, an effective amount of paclitaxel, or a pharmaceutically acceptable salt, prodrug and/or solvate thereof, in combination with an effective amount of homoharringtonine or a pharmaceutically acceptable salt, prodrug and/or solvate thereof.

The present application also includes a use of homoharringtonine, or a pharmaceutically acceptable salt, prodrug and/or solvate thereof, in combination with paclitaxel or a pharmaceutically acceptable salt, prodrug and/or solvate thereof, for improving the efficacy of homoharringtonine for treating a disease, disorder or condition arising from breast cancer in a subject; a use of homoharringtonine, or a pharmaceutically acceptable salt, prodrug and/or solvate thereof, in combination with paclitaxel or a pharmaceutically acceptable salt, prodrug and/or solvate thereof, for the preparation of a medicament for improving the efficacy of homoharringtonine for treating breast cancer in a subject; and the use of homoharringtonine, or a pharmaceutically acceptable salt, prodrug and/or solvate thereof, in combination with paclitaxel or a pharmaceutically acceptable salt, prodrug and/or solvate thereof, for use to improve the efficacy of homoharringtonine for treating breast cancer in a subject.

The present application also includes a method of preventing or reducing breast cancer metastasis comprising administering, to a subject in need thereof, an effective amount of homoharringtonine, or a pharmaceutically acceptable salt, prodrug and/or solvate thereof, in combination with an effective amount of paclitaxel or a pharmaceutically acceptable salt, prodrug and/or solvate thereof.

The present application also includes a use of an effective amount homoharringtonine, or a pharmaceutically acceptable salt, prodrug and/or solvate thereof, in combination with an effective amount of paclitaxel or a pharmaceutically acceptable salt, prodrug and/or solvate thereof, for preventing or reducing breast cancer metastasis in a subject; a use of an effective amount of homoharringtonine, or a pharmaceutically acceptable salt, prodrug and/or solvate thereof, in combination with an effective amount of paclitaxel or a pharmaceutically acceptable salt, prodrug and/or solvate thereof, for preparation of a medicament for preventing or reducing breast cancer metastasis in a subject; and an effective amount of homoharringtonine, or a pharmaceutically acceptable salt, prodrug and/or solvate thereof, in combination with an effective amount of paclitaxel or a pharmaceutically acceptable salt, prodrug and/or solvate thereof, for use to prevent or reduce breast cancer metastasis in a subject.

Classification of breast cancer subtypes is known in the art (see for example, Foulkes et al, N. Engl. J. Med., 2010; 363:1938-1948).

In some embodiments, the breast cancer is a hormone receptor positive breast cancer or a hormone receptor negative breast cancer.

In some embodiments, the breast cancer is a hormone receptor positive breast cancer. In some embodiments, the hormone receptor positive breast cancer is selected from luminal A breast cancer and luminal B breast cancer. In some embodiments, the hormone receptor positive breast cancer is triple positive breast cancer.

In some embodiments, the breast cancer is a hormone receptor negative breast cancer. In some embodiments, the breast cancer is both estrogen receptor positive and progesterone positive cancer. In some embodiments, the breast cancer is an estrogen receptor positive breast cancer. In some embodiments, the breast cancer is a progesterone positive breast cancer.

In some embodiments, the breast cancer is HER2+ breast cancer. In some embodiments, the breast cancer is HER2-breast cancer.

In some embodiments, the hormone receptor negative breast cancer is triple negative breast cancer (TNBC).

Therefore, in some embodiments, the breast cancer is selected from triple-negative breast cancer, triple-positive breast cancer, HER2-negative breast cancer, HER2-positive breast cancer, estrogen receptor positive breast cancer, estrogen receptor negative breast cancer, progesterone receptor positive breast cancer, progesterone receptor negative breast cancer. In some embodiments, the breast cancer is selected from estrogen receptor positive breast cancer, progesterone positive breast cancer, HER2+ breast cancer and triple negative breast cancer.

In some embodiments, the breast cancer is selected from triple negative breast cancer (TNBC), basal-like breast cancer, luminal A breast cancer, luminal B breast cancer, BRCA1 breast cancer, BRCA2 breast cancer, HER2+ breast cancer and combinations thereof. In some embodiments, the breast cancer is selected from triple negative breast cancer (TNBC), basal-like breast cancer and Luminal A breast cancer and combinations thereof. In some embodiments, the breast cancer is luminal A breast cancer. In some embodiments, the breast cancer is selected from triple negative breast cancer (TNBC) or basal-like breast cancer and combinations thereof. In some embodiments, the breast cancer is triple negative breast cancer (TNBC). In some embodiments, the triple negative breast cancer (TNBC) is triple negative metastatic breast cancer.

In some embodiments, the triple negative breast cancer (TNBC) is derived from a TNBC cell line selected from HCC1806, HCC38, HCC1395 and Hs578T.

Accordingly, in some embodiments, the methods and uses of the application are for treating triple negative breast cancer (TNBC) or basal-like breast cancer or combinations thereof, for improving the efficacy of homoharringtonine for treating triple negative breast cancer (TNBC) or basal-like breast cancer or combinations thereof, and for preventing or reducing breast cancer metastasis wherein the breast cancer is treating triple negative breast cancer (TNBC), or basal-like breast cancer or combinations thereof. In some embodiments, the methods and uses of the application are for treating triple negative breast cancer (TNBC), for improving the efficacy of homoharringtonine for treating triple negative breast cancer, and for preventing or reducing triple negative breast cancer (TNBC), metastasis.

Therefore, the present application also includes a method for treating breast cancer, for improving the efficacy of homoharringtonine for treating breast cancer, and for preventing or reducing breast cancer metastasis comprising administering, to a subject in need thereof, a therapeutically effective amount homoharringtonine, or a pharmaceutically acceptable salt, prodrug and/or solvate thereof, in combination with an therapeutically effective amount of paclitaxel or a pharmaceutically acceptable salt, prodrug and/or solvate thereof further in combination with another known agent useful for treatment of breast cancer.

In some embodiments, the breast cancer is triple negative breast cancer (TNBC). In some embodiments, the triple negative breast cancer (TNBC) is triple negative metastatic breast cancer.

The present application also includes a use of an effective amount homoharringtonine, or a pharmaceutically acceptable salt, prodrug and/or solvate thereof, in combination with a effective amount of paclitaxel or a pharmaceutically acceptable salt, prodrug and/or solvate thereof, further in combination with another breast cancer treatment for treating breast cancer, for improving the efficacy of homoharringtonine for treating breast cancer and/or for preventing or reducing breast cancer metastasis. The present application also includes a use of an effective amount homoharringtonine, or a pharmaceutically acceptable salt, prodrug and/or solvate thereof, in combination with a effective amount of paclitaxel or a pharmaceutically acceptable salt, prodrug and/or solvate thereof, further in combination with another breast cancer treatment for the preparation of a medicament for treatment of breast cancer, for improving the efficacy of homoharringtonine for treating breast cancer and/or for preventing or reducing breast cancer metastasis. The application further includes an effective amount homoharringtonine, or a pharmaceutically acceptable salt, prodrug and/or solvate thereof, in combination with an effective amount of paclitaxel or a pharmaceutically acceptable salt, prodrug and/or solvate thereof, further in combination with another breast cancer treatment for use in treating breast cancer, for improving the efficacy of homoharringtonine for treating breast cancer and/or for preventing or reducing breast cancer metastasis.

In some embodiments, the additional breast cancer treatment is selected from one or more of radiotherapy, chemotherapy, targeted therapies such as antibody therapies, small molecule therapies such as tyrosine-kinase inhibitors therapies, glutaminase inhibitors (GLS1 inhibitors), immunotherapy, hormonal therapy, surgical intervention, radiation therapy and anti-angiogenic therapies.

In some embodiments, the chemotherapy comprises the use or administration of one or more chemotherapeutic agents. In some embodiments, the one or more chemotherapeutic agents are selected from platinum drugs (such as cisplatin and carboplatin), doxorubicin, epirubicin, capecitabine, cyclophosphamide, eribulin, fluorouracil, gemcitabine, ixabepilone, methotrexate and vinorelbine and combinations thereof.

In some embodiments, the hormonal therapy comprises the use or administration of one or more of tamoxifen, aromatase inhibitors, and gonadotropin or luteinizing releasing hormone (GnRH or LHRH) agonist drugs (such as goserelin and leuprolide).

In some embodiments, the targeted therapy comprises the use of administration of one or more of pertuzumab, trastuzumab, hyaluronidase-zzxf, neratinib, ado-trastuzumab emtansine, olapari, abemaciclib, alpelisib, palbociclib, ribciclib, lapatinib, tucatinib, sacituxumab, entrectinib, larotrectinib, olaparib and talazoparib.

In some embodiments, immunotherapy comprises use or administration of pembrolizumab.

In some embodiments, the additional breast cancer treatment is conducted or performed according to methods known in the art for the specific treatment, including dosing regimens and/or amounts.

In some embodiments the subject is a mammal. In some embodiments, the subject is human.

In some embodiments, the “subject in need thereof” is a subject having breast cancer. In some embodiments, the subject in need thereof is a subject having breast cancer and the breast cancer is in remission.

In the context of treating breast cancer, an effective amount of homoharringtonine, or a salt, prodrug and/or solvate thereof, and/or paclitaxel, or a salt, prodrug and/or solvate thereof, is an amount that, for example, treats the breast cancer compared to treatment of breast cancer without administration of homoharringtonine, or a salt, prodrug and/or solvate thereof, and paclitaxel, or a salt, prodrug and/or solvate thereof. Further, in the context of improving the efficacy of homoharringtonine, or a salt, prodrug and/or solvate thereof, for the treatment of breast cancer an effective amount of paclitaxel, or a salt, prodrug and/or solvate thereof, is, for example, an amount that, improves the efficacy of homoharringtonine, or a salt, prodrug and/or solvate thereof, for the treatment of breast cancer compared to without administration of paclitaxel, or a salt, prodrug and/or solvate thereof. Similarly, in the context of preventing or reducing breast cancer metastasis an effective amount of homoharringtonine, or a salt, prodrug and/or solvate thereof, and/or paclitaxel, or a salt, prodrug and/or solvate thereof, is an amount that, for example, reduces breast cancer metastasis compared to breast cancer metastasis without administration of homoharringtonine, or a salt, prodrug and/or solvate thereof, and paclitaxel, or a salt, prodrug and/or solvate thereof.

The Applicants have found that the combination of homoharringtonine and paclitaxel is very effective in killing breast cancer cells. Further, the synergy of homoharringtonine and paclitaxel for killing breast cancer cells was observed at doses that were considerably lower than the EC₅₀ values of the individual drugs when used alone. Therefore, in some embodiments, the effective amount is a synergistically effective amount. Therefore, the present application also includes administering to a subject in need thereof and use of a synergistically effective amount of homoharringtonine, or a pharmaceutically acceptable salt, prodrug and/or solvate thereof, in combination with a synergistically effective amount of homoharringtonine or a pharmaceutically acceptable salt, prodrug and/or solvate thereof in the methods and uses of the application described herein.

In some embodiments, the synergistically effective amount that will be administered or used is determined based on the species being treated, the subject's age, the subject weight, method of administration, general symptoms of the subject, complications, and/or the severity of the symptoms being treated. In some embodiments, the synergistically effective amount will vary from subject to subject depending on such factors but can be determined by one of ordinary skill in the art using routine methods. In some embodiments, paclitaxel, or a salt, prodrug and/or solvate thereof, and homoharringtonine, or a salt, prodrug and/or solvate thereof, are administered initially in a suitable amount that may be adjusted as required, depending on the clinical response.

In some embodiments, homoharringtonine, or a salt, prodrug and/or solvate thereof, and/or paclitaxel, or a salt, prodrug and/or solvate thereof, are administered or used according to treatment protocol that is known for homoharringtonine, or a salt, prodrug and/or solvate thereof, and/or paclitaxel, or a salt, prodrug and/or solvate thereof for the chemotherapeutic agents in the treatment of cancer.

The Applicants have found that the combination of homoharringtonine and paclitaxel is very effective in killing breast cancer cells at doses that were considerably lower than the EC₅₀ values of individual drugs when used alone. Accordingly, in some embodiments, the dosage of homoharringtonine, or a salt, prodrug and/or solvate thereof and the dosage of paclitaxel, or a salt, prodrug and/or solvate thereof, is equal to or less than the dosage of each agent when used alone. Therefore, in some embodiments, the homoharringtonine, or a salt, prodrug and/or solvate thereof and the paclitaxel, or a salt, prodrug and/or solvate thereof, are administered or used at dosages that are equal to or less than a dosage of the homoharringtonine, or a salt, prodrug and/or solvate thereof and the paclitaxel, or a salt, prodrug and/or solvate thereof when each is used alone. In some embodiments, the dosage of homoharringtonine, or a salt, prodrug and/or solvate thereof, is equal to or less than the dosage of such agent when used alone or without paclitaxel, or a salt, prodrug and/or solvate thereof. In some embodiments, the dosage of paclitaxel, or a salt, prodrug and/or solvate thereof, is equal to or less than the dosage of such agent when used alone or without homoharringtonine, or a salt, prodrug and/or solvate thereof. Such dosages are known to or readily determined by those skilled in the art.

In some embodiments, the homoharringtonine, or a salt, prodrug and/or solvate thereof and/or the paclitaxel, or a salt, prodrug and/or solvate thereof, are administered or used at dosages that are equal or at least about 5%, at least about 10%, at least about 20%, at least about 30%, at least about 40%, or at least about 50% lower than a dosage of the homoharringtonine, or a salt, prodrug and/or solvate thereof and/or the paclitaxel, or a salt, prodrug and/or solvate thereof when each is used alone. In some embodiments, homoharringtonine, or a salt, prodrug and/or solvate thereof, is administered or used at dosages that is equal to or at least about 5%, at least about 10%, at least about 20%, at least about 30%, at least about 40%, or at least about 50% lower less than a dosage of such agent when used alone or without paclitaxel, or a salt, prodrug and/or solvate thereof. In some embodiments, paclitaxel, or a salt, prodrug and/or solvate thereof is administered or used at dosages that is equal to or at least about 5%, at least about 10%, at least about 20%, at least about 30%, at least about 40%, or at least about 50% lower less than the dosage of such agent when used alone or without homoharringtonine, or a salt, prodrug and/or solvate thereof.

In some embodiments, the frequency of administration or use of homoharringtonine, or a salt, prodrug and/or solvate thereof and/or the paclitaxel, or a salt, prodrug and/or solvate thereof that achieves a therapeutic effect (e.g., treats breast cancer, improves the efficacy of homoharringtonine for treating breast cancer and/or for preventing or reduces breast cancer metastasis) is at least about 5%, at least about 10%, at least about 15%, at least about 20%, at least about 25%, at least about 30%, at least about 40%, or at least about 50% lower when homoharringtonine or a pharmaceutically acceptable salt, prodrug and/or solvate thereof is administered in combination with paclitaxel, or a pharmaceutically acceptable salt, prodrug and/or solvate thereof than when the homoharringtonine or a pharmaceutically acceptable salt, prodrug and/or solvate thereof is administered or used alone.

Treatment methods comprise administering to a subject paclitaxel, or a salt, prodrug and/or solvate thereof, and homoharringtonine, or a salt, prodrug and/or solvate thereof, and optionally consists of a single administration, or alternatively comprises a series of administrations. In some embodiments, homoharringtonine, or a salt, prodrug and/or solvate thereof, and/or paclitaxel, or a salt, prodrug and/or solvate thereof, are administered or used one, two, three or four times a year. In some embodiments, homoharringtonine, or a salt, prodrug and/or solvate thereof, and/or paclitaxel, or a salt, prodrug and/or solvate thereof, are administered or used at least once a week. In some embodiments, homoharringtonine, or a salt, prodrug and/or solvate thereof, and/or paclitaxel, or a salt, prodrug and/or solvate thereof, are administered or used from about one time per two weeks, three weeks or one month. In some embodiments, the homoharringtonine, or a salt, prodrug and/or solvate thereof, and/or paclitaxel, or a salt, prodrug and/or solvate thereof, are administered or used about one time per week to about once daily. In another embodiment, the homoharringtonine, or a salt, prodrug and/or solvate thereof, and/or paclitaxel, or a salt, prodrug and/or solvate thereof, are administered or used 1, 2, 3, 4, 5 or 6 times daily. In some embodiments, paclitaxel, or a salt, prodrug and/or solvate thereof, is administered or used orally 3 times per week. In some embodiments, paclitaxel, or a salt, prodrug and/or solvate thereof, is administered or used intravenously about one time every three weeks. In some embodiments, paclitaxel, or a salt, prodrug and/or solvate thereof, is administered or used intravenously about three times per week every three weeks. In some embodiments, the homoharringtonine, or a salt, prodrug is administered or used subcutaneously about every 12 hours for about 2 weeks. The length of the treatment period depends on a variety of factors, such as the severity of the disease, disorder or condition, the age of the subject, the concentration and/or the activity of homoharringtonine, or a salt, prodrug and/or solvate thereof, and/or paclitaxel, or a salt, prodrug and/or solvate thereof, or a combination thereof. It will also be appreciated that the effective dosage of homoharringtonine, or a salt, prodrug and/or solvate thereof, and/or paclitaxel, or a salt, prodrug and/or solvate thereof used for the treatment or prevention may increase or decrease over the course of a particular treatment regime. Changes in dosage may result and become apparent by standard diagnostic assays known in the art.

In some embodiment, the dosages of homoharringtonine, or a salt, prodrug and/or solvate thereof or paclitaxel, or a salt, prodrug and/or solvate thereof are formulated for oral or intravenous administration or use. As a representative example, oral dosages of paclitaxel, or a salt, prodrug and/or solvate thereof, is about 205 mg/m² or less, or about 205 mg/m², about 200 mg/m², about 195 mg/m², about 190 mg/m², about 185 mg/m², about 180 mg/m², about 175 mg/m², about 170 mg/m², about 160 mg/m² or about 150 mg/m² about 150 mg/m² to about 205 mg/m² or about 175 mg/m² to about 205 mg/m². In some embodiments, oral dosages of paclitaxel, or a salt, prodrug and/or solvate thereof, is about 205 mg/m². As a representative example, intravenous dosages of paclitaxel, or a salt, prodrug and/or solvate thereof, is about 175 mg/m² or less, or about 175 mg/m², about 170 mg/m², about 160 mg/m², about 150 mg/m², about 125 mg/m² or about 125 mg/m² to about 175 mg/m² or about 150 mg/m² to about 175 mg/m². In some embodiments, oral dosages of paclitaxel, or a salt, prodrug and/or solvate thereof, is about 175 mg/m². In some embodiment, the dosages of homoharringtonine, or a salt, prodrug and/or solvate thereof are formulated for subcutaneous administration or use. As a representative example, subcutaneous dosages of homoharringtonine, or a salt, prodrug and/or solvate thereof, is about 1.25 mg/m² or less, or about 1.25 mg/m², about 1.20 mg/m² about 1.15 mg/m² about 1.10 mg/m² about 1.05 mg/m² about 1.00 mg/m² or about 1.0 mg/m² to about 1.25 mg/m², about 1.00 mg/m² or about 1.25 mg/m² to about 1.00 mg/m² or about 1.25 mg/m² to about 1.15 mg/m². In some embodiments, the paclitaxel, or a salt, prodrug and/or solvate thereof, is administered or used in combination with an absorption enhancing agent. In some embodiments, the absorption enhancing agent of the paclitaxel, or a salt, prodrug and/or solvate thereof is encequidar. In some embodiment, the dosages of homoharringtonine, or a salt, prodrug and/or solvate thereof are formulated for intravenous administration or use. As a representative example, intravenous dosages of homoharringtonine, or a salt, prodrug and/or solvate thereof, is about 2.5 mg/m² or less, 2.0 mg/m² or less, 1.25 mg/m² or less, or about 2.5 mg/m², about 2.25 mg/m², about 2.0 mg/m² about 1.5 mg/m², about 1.25 mg/m², about 1.20 mg/m² about 1.15 mg/m² about 1.10 mg/m² about 1.05 mg/m² about 1.00 mg/m² or about 2.5 mg/m² to about 1.25 mg/m², about 2.0 mg/m² to about 1.25 mg/m².

In some embodiments, suitable pharmaceutically acceptable acid addition salts are prepared from an inorganic acid or an organic acid. Examples of such inorganic acids include, without limitation, hydrochloric, hydrobromic, hydroiodic, nitric, carbonic, sulfuric and phosphoric acid. In some embodiments, the homoharringtonine salt is homoharringtonine hydrochloride. Examples of suitable organic acids include, for example, aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. In some embodiments, the homoharringtonine salt is a homoharringtonine diacid salt. For example, the homoharringtonine salt is fumaric, maleic, citramalic, malic, tartaric, tartronic, succinic, itaconic, or citric acid salt of homoharringtonine.

Formation of a pharmaceutically-acceptable salt of the compounds may be achieved using standard techniques. For example, a neutral compound is treated with an acid or base in a suitable solvent and the formed salt is isolated by filtration, extraction or any other suitable method.

Examples of suitable solvate solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Various forms, including crystal forms and solvates, of paclitaxel are described, for example, in Harper, J. K., et al, Crystal Growth & Design 2005 5 (5), 1737-1742, Heider E. et al., 2007 Physical Chemistry Chemical Physics 9 (46): 6083-97, EP0717041A1, US20030144344A1 and WO2014152360A1 the contents of which are incorporated herein by reference.

Various crystal forms and salts of homoharringtonine are described, for example, in WO2002074776A2, US20190161493A1 and WO2015101628A1 the contents of which are incorporated herein by reference.

Various prodrugs of paclitaxel are described, for example, in Meng Z., et al., Int J Mol Sci. 2016 May; 17 (5), 796, Skwarczynski, M. et al., J. Med. Chem. 2006, 49, 25, 7253-7269, Shan L. et al., RSC Adv., 2016, 6, 77987-77998, Bio M. et al., Bioorganic & Medicinal Chemistry Letters, 29, 12, 2019, 1537-1540, J. Mater. Chem. B, 2021, 9, 2308-2313, and Dhanikula A. B and Ramesh P., 2 (1), 2005, p75-91, the contents of which are incorporated herein by reference.

Prodrugs may be prepared, for example, by acylating available hydroxy or amino groups using an activated acid in the presence of a base, and optionally, in inert solvent (e.g. an acid chloride in pyridine). Similarly, available carboxylic acid groups may be converted to ester groups using known chemistry, for example, by activation in the presence of base and reaction with suitable groups containing a nucleophile. Some common esters which have been utilized as prodrugs are phenyl esters, aliphatic (C1-C24) esters, acyloxymethyl esters, carbamates and amino acid esters.

The homoharringtonine, or a salt, prodrug and/or solvate thereof, and/or paclitaxel or a salt, prodrug and/or solvate thereof are administered to a subject, or used, in a variety of forms depending on the selected route of administration, as will be understood by those skilled in the art. In some embodiments, the homoharringtonine, or a salt, prodrug and/or solvate thereof, and/or paclitaxel or a salt, prodrug and/or solvate thereof are administered to the subject, or used, by oral (including sublingual and buccal) or parenteral (including, intravenous, intraperitoneal, subcutaneous, intramuscular, transepithelial, nasal, intrapulmonary, intrathecal, rectal, topical, patch, pump and transdermal) administration and the antibiotic formulated accordingly. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringability exists.

In some embodiments, parenteral administration is by continuous infusion over a selected period of time. Solutions suitable for parenteral administration are prepared by known methods by a person skilled in the art. For example, paclitaxel or a salt, prodrug and/or solvate thereof, or homoharringtonine, or a salt, prodrug and/or solvate thereof, is prepared in water optionally mixed with a surfactant such as hydroxypropylcellulose. Dispersions are also prepared in glycerol, liquid polyethylene glycols, DMSO and mixtures thereof with or without alcohol, and in oils. Under ordinary conditions of storage and use, these preparations contain a preservative to prevent the growth of microorganisms.

Compositions for nasal administration are conveniently formulated as aerosols, drops, gels or powders. Aerosol formulations typically comprise a solution or fine suspension of the active substance in a physiologically acceptable aqueous or non-aqueous solvent and are usually presented in single or multidose quantities in sterile form in a sealed container, which take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it contains a propellant which is, for example, a compressed gas such as compressed air or an organic propellant such as fluorochlorohydrocarbon. In some embodiments, the aerosol dosage forms take the form of a pump-atomizer.

Compositions suitable for buccal or sublingual administration include tablets, lozenges, and pastilles, wherein the active ingredient is formulated with a carrier such as sugar, acacia, tragacanth, gelatin and/or glycerine. Compositions for rectal administration are conveniently in the form of suppositories containing a conventional suppository base such as cocoa butter.

In another embodiment, homoharringtonine, or a salt, prodrug and/or solvate thereof, and/or paclitaxel or a salt, prodrug and/or solvate thereof are orally administered, for example, with an inert diluent or with an assimilable edible carrier, or it is enclosed in hard or soft shell gelatin capsules, or it is compressed into tablets, or it is incorporated directly with the food of a diet. For oral administration, homoharringtonine, or a salt, prodrug and/or solvate thereof, and/or paclitaxel or a salt, prodrug and/or solvate thereof are incorporated with excipients and used in the form of, for example, ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., molecular sieving type particles, or, a fine hollow permeable fiber bundle, or chopped hollow permeable fibers, agglomerated or held in a fibrous packet. In some embodiments, timed-release compositions are, formulated, as liposomes or those wherein the active compounds are protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of lipids, such as cholesterol, stearylamine or phosphatidylcholines.

It is also possible to freeze-dry homoharringtonine, or a salt, prodrug and/or solvate thereof, and/or paclitaxel or a salt, prodrug and/or solvate thereof are, and use the lyophilizate obtained, for example, for the preparation of products for injection.

In some embodiments, homoharringtonine, or a salt, prodrug and/or solvate thereof, and/or paclitaxel or a salt, prodrug and/or solvate thereof are coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. In a further embodiment, paclitaxel or a salt, prodrug and/or solvate thereof, or homoharringtonine, or a salt, prodrug and/or solvate thereof, is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Homoharringtonine, or a salt, prodrug and/or solvate thereof, and/or paclitaxel or a salt, prodrug and/or solvate thereof are used in combination with each other. Homoharringtonine, or a salt, prodrug and/or solvate thereof, and/or paclitaxel or a salt, prodrug and/or solvate thereof, are either used or administered separately in time and/or in mode of administration (i.e. different administration routes) or they are administered together in the same pharmaceutical preparation.

In one embodiment, homoharringtonine, or a salt, prodrug and/or solvate thereof, and/or paclitaxel or a salt, prodrug and/or solvate thereof, are used or administered separately in time and/or in mode of administration. For example, paclitaxel or a salt, prodrug and/or solvate thereof, is administered by injection and homoharringtonine, or a salt, prodrug and/or solvate thereof, is administered orally. In another example, paclitaxel, or a salt, prodrug and/or solvate thereof, is administered orally and homoharringtonine, or a salt, prodrug and/or solvate thereof, is administered by injection. In a further example, both paclitaxel, or a salt, prodrug and/or solvate thereof, and homoharringtonine, or a salt, prodrug and/or solvate thereof, are administered orally or by injection. When paclitaxel, or a salt, prodrug and/or solvate thereof, and homoharringtonine, or a salt, prodrug and/or solvate thereof, are used or administered separately in time and/or in mode of administration, paclitaxel, or a salt, prodrug and/or solvate thereof, is administered, or used, either before or after administration, or use, of homoharringtonine, or a salt, prodrug and/or solvate thereof.

In another embodiment, paclitaxel, or a salt, prodrug and/or solvate thereof, and homoharringtonine, or a salt, prodrug and/or solvate thereof, are administered contemporaneously. As used herein, “contemporaneous administration” of two substances to a subject means providing paclitaxel, or a salt, prodrug and/or solvate thereof, and homoharringtonine, or a salt, prodrug and/or solvate thereof, so that they are both biologically active in the subject at the same time. The exact details of the administration will depend on the pharmacokinetics of paclitaxel, or a salt, prodrug and/or solvate thereof, and homoharringtonine, or a salt, prodrug and/or solvate thereof, in the presence of each other, and can include administering paclitaxel, or a salt, prodrug and/or solvate thereof, and homoharringtonine, or a salt, prodrug and/or solvate thereof, within a few hours of each other, or even administering paclitaxel, or a salt, prodrug and/or solvate thereof, and homoharringtonine, or a salt, prodrug and/or solvate thereof, within 24 hours or greater of administration of the other, if the pharmacokinetics are suitable. Design of suitable dosing regimens is routine for one skilled in the art.

In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, compounds of the present application are administered with another therapeutic agent simultaneously or sequentially in separate unit dosage forms or together in a single unit dosage form. Accordingly, the present application provides a single unit dosage form comprising the compounds, an additional therapeutic agent, and a pharmaceutically acceptable carrier.

In some embodiments, paclitaxel, or a salt, prodrug and/or solvate thereof, and homoharringtonine or a salt, prodrug and/or solvate thereof, are administered to a subject in a single composition or formulation.

In another embodiment of the present application, paclitaxel, or a salt, prodrug and/or solvate thereof, and homoharringtonine, or a salt, prodrug and/or solvate thereof, are administered to a subject in a non-contemporaneous fashion.

In a further embodiment of the present application, paclitaxel, or a salt, prodrug and/or solvate thereof, and homoharringtonine, or a salt, prodrug and/or solvate thereof, are administered to the subject in a contemporaneous fashion followed by, or alternating with, administration in a non-contemporaneous fashion.

### III. Compositions and Kits of the Application

The present application also includes a pharmaceutical composition comprising homoharringtonine or a pharmaceutically acceptable salt, prodrug and/or solvate thereof and paclitaxel or a pharmaceutically acceptable salt, prodrug and/or solvate thereof wherein the homoharringtonine and paclitaxel are present in amounts that are effective to treat breast cancer, for improving the efficacy of homoharringtonine for treating breast cancer and/or for preventing or reducing breast cancer metastasis.

In some embodiments, the amounts that are effective are synergistic effective amounts. Therefore, the present application also includes compositions and pharmaceutical compositions comprising homoharringtonine or a pharmaceutically acceptable salt, prodrug and/or solvate thereof and paclitaxel or a pharmaceutically acceptable salt, prodrug and/or solvate thereof wherein the homoharringtonine and paclitaxel are present in amounts that are synergistically effective for treating breast cancer, for improving the efficacy of homoharringtonine for treating breast cancer or for preventing or reducing breast cancer metastasis.

Accordingly, in some embodiments, the amounts that are synergistically effective for treating breast cancer, for improving the efficacy of homoharringtonine for treating breast cancer or for preventing or reducing breast cancer metastasis of homoharringtonine, or a salt, prodrug and/or solvate thereof are equal to or less than the effective amount of homoharringtonine or a pharmaceutically acceptable salt, prodrug and/or solvate thereof and/or paclitaxel or a pharmaceutically acceptable salt, prodrug and/or solvate thereof when used alone. In some embodiments, the amounts that are synergistically effective for treating breast cancer, for improving the efficacy of homoharringtonine for treating breast cancer or for preventing or reducing breast cancer metastasis of homoharringtonine, or a salt, prodrug and/or solvate thereof, is equal to or less than the dosage of such agent when used alone or without paclitaxel, or a salt, prodrug and/or solvate thereof. In some embodiments, the amounts that are synergistically effective for treating breast cancer, for improving the efficacy of homoharringtonine for treating breast cancer or for preventing or reducing breast cancer metastasis of paclitaxel, or a salt, prodrug and/or solvate thereof, is equal to or less than the dosage of such agent when used alone or without homoharringtonine, or a salt, prodrug and/or solvate thereof.

In some embodiments, the amounts that are synergistically effective for treating breast cancer, for improving the efficacy of homoharringtonine for treating breast cancer or for preventing or reducing breast cancer metastasis are equal or at least about 5%, at least about 10%, at least about 20%, at least about 30%, at least about 40%, or at least about 50% lower than the effective amounts of the homoharringtonine, or a salt, prodrug and/or solvate thereof and/or the paclitaxel, or a salt, prodrug and/or solvate thereof when each is used alone.

The present application also includes a kit for the treatment breast cancer, the kit comprising paclitaxel or a pharmaceutically acceptable salt, prodrug and/or solvate thereof and homoharringtonine or a pharmaceutically acceptable salt, prodrug and/or solvate thereof and optionally instructions for administration of paclitaxel or a pharmaceutically acceptable salt, prodrug and/or solvate thereof and homoharringtonine, or a pharmaceutically acceptable salt and/or solvate thereof, to a subject in need thereof.

The present application also includes a kit for the treatment of a breast cancer, the kit comprising: homoharringtonine or a pharmaceutically acceptable salt and/or solvate thereof; and instructions for administration of homoharringtonine, or a pharmaceutically acceptable salt and/or solvate thereof, to a subject being administered paclitaxel for treating breast cancer.

The present application also includes a kit for the treatment of a breast cancer, the kit comprising: paclitaxel or a pharmaceutically acceptable salt and/or solvate thereof; and instructions for administration of paclitaxel, or a pharmaceutically acceptable salt and/or solvate thereof, to a subject being administered homoharringtonine for treating breast cancer.

The present application also includes a kit for improving the efficacy of homoharringtonine for the treatment of breast cancer, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

The present application also includes a kit for improving the efficacy of homoharringtonine for the treatment breast cancer, the kit comprising homoharringtonine, or a pharmaceutically acceptable salt and/or solvate thereof; and instructions for administration of homoharringtonine, or a pharmaceutically acceptable salt and/or solvate thereof, to a subject being administered paclitaxel for the treatment of breast cancer.

The present application also includes a kit for preventing or reducing breast cancer metastasis, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

In some embodiments, the breast cancer is selected from triple negative breast cancer, basal-like breast cancer, luminal A breast cancer, luminal B breast cancer, BRCA1 breast cancer, BRCA2 breast cancer, HER2+ breast cancer and combinations thereof. In some embodiments, the breast cancer is selected from triple negative breast cancer, basal-like breast cancer and luminal A breast cancer and combinations thereof. In some embodiments, the breast cancer is luminal A breast cancer. In some embodiments, the breast cancer is selected from triple negative breast cancer or basal-like breast cancer and combinations thereof. In some embodiments, the breast cancer is triple negative breast cancer.

In some embodiments, homoharringtonine or a pharmaceutically acceptable salt, prodrug and/or solvate thereof and paclitaxel or a pharmaceutically acceptable salt, prodrug and/or solvate thereof, in the compositions and kits of the present application are formulated as separate pharmaceutical compositions, for separate administration to, or use in, subjects. In this embodiment, the separate pharmaceutical compositions are formulated independently of each other and in accordance with the desired mode of administration for each active.

In some embodiments, homoharringtonine or a pharmaceutically acceptable salt and/or solvate thereof is formulated for administration, or use, by oral delivery or for delivery by injection. In another embodiment, paclitaxel, or a pharmaceutically acceptable salt and/or solvate thereof is formulated for administration, or use, by oral delivery or for delivery by injection.

In some embodiments, homoharringtonine, or a pharmaceutically acceptable salt and/or solvate thereof, and the paclitaxel or a pharmaceutically acceptable salt and/or solvate thereof in the compositions and kits of the present application are formulated as a single pharmaceutical composition, for combined, simultaneous administration to, or use in, subjects. In some embodiments, the single pharmaceutical composition is formulated for administration, or use, by oral delivery or by injection.

The following non-limiting examples are illustrative of the present application:

### EXAMPLES

### Example 1

### Cell Lines and Cell Culture

Cell lines used included TNBC cell lines (HCC1806, BT549, HCC1395, HCC38, Hs578T, MDA-MB-157, MDA-MB-436), luminal A cell lines (MCC1428, MCF7, T47D, ZR-75-1) and non-tumorigenic breast cell line (MCF10A), all obtained from the American Type Culture Collection (ATCC). Cells were authenticated by the supplier (http://www.ATCC.org) and cultured as recommended by ATCC for less than six months from the time of resuscitation. HCC1806, HCC1428 and T47D cells stably expressing triple hemagglutinin (HA)-tagged CREB3L1 (HA-CREB3L1) have been described and characterized previously [14].

To visualize and count cells for the primary high-throughput drug screen, HCC1806 cells and HCC1806+HACREB3L1 cells were labeled by the stable expression of red-fluorescent protein (RFP). Each cell line was transduced with an RFP-encoding lentivirus (pLJM5-RFP-hygro) and selected for 2 weeks in hygromycin (12.5 μg/mL; 10687-010, Invitrogen). The RFP labeled cells growing in hygromycin-containing media grew more slowly than their unlabeled counterparts, but had similar doubling times to each other, though their plating efficiencies were different. There was no difference in the proliferation rate between HCC1806 and HCC1806+HACREB3L1 cells (both RFP-expressing and non-RFP-expressing), though they also had different plating efficiencies.

### Primary Drug Screen

A high-throughput drug screen of an FDA-approved drug library (1,818 compounds; TargetMol, L1000) was carried out on HCC1806+HACREB3L1 cells at the Phenogenomic Imaging Centre of Saskatchewan. Briefly, in parallel, the RFP-labeled cell types were seeded into 384-well black-walled plates (142761, NUNC), HCC1806 cells (1300 cells/well) and HCC1806+HA-CREB3L1 (2600 cells/well) in a total volume of 50 μL/well. Different starting cell numbers were used to take into account differences in their plating efficiencies noted above. Cells were allowed to attach and grow at 37° C. and 5% CO. The following day drugs were added to each well (0.2 μL; 250 μM stock in dimethyl sulfoxide (DMSO)) giving a final concentration of 1 μM using a Biomek FX® liquid handling system (A31843). Control DMSO wells were also included to control for impacts independent of the test drug. Live cells were imaged both prior to and after drug addition, and then each day for a total of 4 days using an automated imaging fluorescence microscope system, ImageXpress® Micro XLS Widefield (Molecular Devices). The number of RFP-labeled cells remaining attached to the well were counted over time. After 4 days of drug treatment, cell viability (%) was determined for each drug test well relative to the corresponding DMSO control well.

### Secondary Drug Screen

Drugs selected from the primary screen (47) were independently sourced and reconstituted using the appropriate solvent (DMSO, dimethylformamide (DMF), ethanol (EtOH) or phosphate buffered saline (PBS)) as indicated by the supplier (Cedarlane). Drugs were generally reconstituted to 10 mM, except for a few that had a lower solubility, and all reconstituted drugs were stored at −80° C. To use, drugs were diluted into media (to 1 μM) to maintain a low solvent concentration (<0.1%) and solvent control samples were generated and tested in parallel.

For secondary drug testing, unlabeled HCC1806 (500 cells/well) and HCC1806+HA-CREB3L1 cells (600 cells/well) were seeded in triplicate wells in sterile 384-well black-walled optical bottom plate (6007558, PerkinElmer) using an ASSIST PLUS pipetting robot and a 16-channel VIAFLO pipette (4505 and 4642, INTEGRA Biosciences AG) in 50 μL. The doubling time of HCC1806 cells was not significantly impacted by HA-CREB3L1 expression [14], though their initial plating efficiencies differed slightly. Cells were allowed to attach and grow at 37° C. and 5% CO. The following day the media was carefully removed from each well and replaced with drug-containing media in triplicate wells, again using the ASSIST PLUS. After 4 days of drug treatment cells were stained with Hoechst and ImageIT® Dead Green to quantify total and live cells as detailed below.

### Cell Staining and Viability Determination

To quantify total and dead cells, they were stained with media containing Hoechst 33324 dye (5 μM; ThermoFisher Scientific, 62249) and ImageIT® Dead Green dye (100 nM; ThermoFisher Scientific, 110291) for 30 min at 37rC with 5% CO. Images of each well of the 384-well plates were acquired using a Thermo Scientific® CellInsight® CX7 High Content Screening (HCS) Platform. Nine fields of view were captured using a 10×(0.4 NA) air objective lens. Images were analyzed using Thermo Scientific HCS Studio® 3 Cell Analysis Software using the Spot Detector bio-application (Cellomics®). Total cells were identified and counted by their Hoechst-stained nuclei at 386 nm (blue) channel 1 (Ch1) and dead cells were quantified from the ImageIT® Dead Green on the 485 nm wavelength (green) channel 2 (Ch2). The total live cell count for each well containing a drug (determined from Ch1), was normalized to the total live cell count for each control well to account for any dead cells that had lifted off of the plate in test wells. The test well total live cell count was divided by the average of the corresponding solvent control total live cell count, giving the percentage cell viability (% viability). The mean±standard error of the mean (SEM) of the % viability of each set of triplicate wells was determined.

### EC₅₀ Determinations-Drug Titration Experiments

Drug titration experiments were carried out as described for the secondary drug screens, except a range of drug concentrations were tested. Typically, drugs were serially diluted in media 1:3 over the range of drug concentrations as noted in the text and figure legends (e.g., 0-333 nM, 0-2!M, 0-9 μM, 0-50 μM), using the appropriate solvent control wells. Some drugs had to be further diluted to even lower concentrations, typically using 1:2 dilutions to best define the dose response curve. EC values were determined using PRISM software (GraphPad, v9.2.0) using non-linear curve fit. A student's t-test was used to determine statistically significant differences between the EC values for the two cell lines. Significance was defined as p<0.05. EC values of drugs that were more cytotoxic to the CREB3L1 deficient cells were determined from three independent experiments, each typically containing triplicate measurements. EC values of drugs that were equally or less cytotoxic to CREB3L1 deficient cells were determined from one experiment, typically containing triplicate measurements.

For drug titration experiments involving additional cell lines, plating efficiencies were determined for each cell line so that an appropriate number of cells were used to have significant numbers of cells to count in control (solvent only) wells over the 5 day experiment.

### Synergy Experiments

Combination drug titration experiments were performed to determine potential selective cytotoxic sensitivity and synergy of the top 4 compounds with known chemotherapeutic reagents, doxorubicin (A3966, ApexBio, made up in DMSO) and paclitaxel (10461, Cayman Chemical, made up in DMSO) in the paired TNBC cell lines CREB3L1-deficient HCC1806 cells and CREB3L1 re-expressing HCC1806+HACREB3L1 cells. A cost-effective and robust cross-design format was used which combines a background drug with a foreground drug [16]. The background drug is used at its EC value, whereas the foreground drug is tested across a range of doses. Each drug in the combination is tested as both a background drug and a foreground drug. Cells were plated, treated and cell viability was assessed as described previously. Results from 3 titrations were analyzed for each drug combination.

To determine the possible synergy of the combined drugs SynergyFinder software (version 2.0.11) was used with the default settings in R (version 3.6.1) [17]. The SynergyFinder package adjusted the % Viability (response) values input to % Cytotoxicity values. There were no other adjustments made to the data. Both monotherapy and combination data were input into the program. A delta score of 0 indicated no interaction or additivity (white in the synergy landscape). A negative delta score indicated antagonism (green in the synergy landscape). A positive delta score indicated synergy (red in the synergy landscape), with values >10 indicating significant synergy.

### Statistical Analyses

All results were expressed as the mean±standard errors from at least 3 independent experiments, unless otherwise indicated. The significance of changes was assessed by the application of a student's t-test significance considered at p<0.05 (Microsoft Excel v16.56).

### Results

### High-Throughput Drug Screen and EC₅₀ Determinations

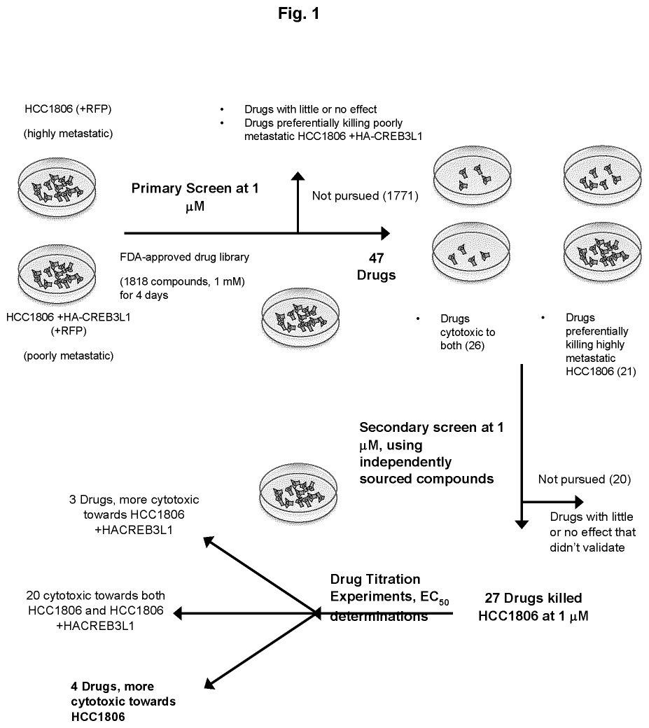

To identify drugs that are selectively cytotoxic towards more highly metastatic TNBC cells a matched pair of cell lines was used: highly metastatic CREB3L1-deficient HCC1806 cells and poorly metastatic HCC1806+HA-CREB3L1 cells [14]. To help visualize and count cells, each cell line was stably transfected to express red fluorescent protein (RFP). A high-throughput drug screen was performed using an FDA-approved library of 1,818 compounds at an initial test concentration of 1 UM for 4 days of drug treatment (FIG. 1). Two groups of drugs were advanced to secondary screening: 1) drugs that showed 40% or more cytotoxicity towards the metastatic CREB3L1-deficient HCC1806 cells (21 drugs), and 2) drugs that killed both cell lines with similar efficacy at this single test concentration (1 μM) in case they might show selective killing of HCC1806 when tested at lower concentrations (26 drugs). A total of 47 drugs were identified as either more cytotoxic towards CREB3L1-deficient HCC1806 cells, as compared to HCC1806+HA-CREB3L1 cells, or very cytotoxic towards both cell lines.

The 47 compounds identified from the drug library were purchased from independent commercial sources and a validation experiment was carried out. All subsequent cytotoxicity assays used non-RFP-labeled cells and counted total cells (Hoechst 33324 dye), subtracting dead cells (ImageIT Dead Green dye), to determine cell viability after 4 days of drug treatment. The initial validation experiment tested the cytotoxicity of each of the 47 drugs towards HCC1806+HA-CREB3L1 cells at 1 μM with triplicate measurements, with 27 drugs validated as cytotoxic to one or both cell lines (FIG. 1).

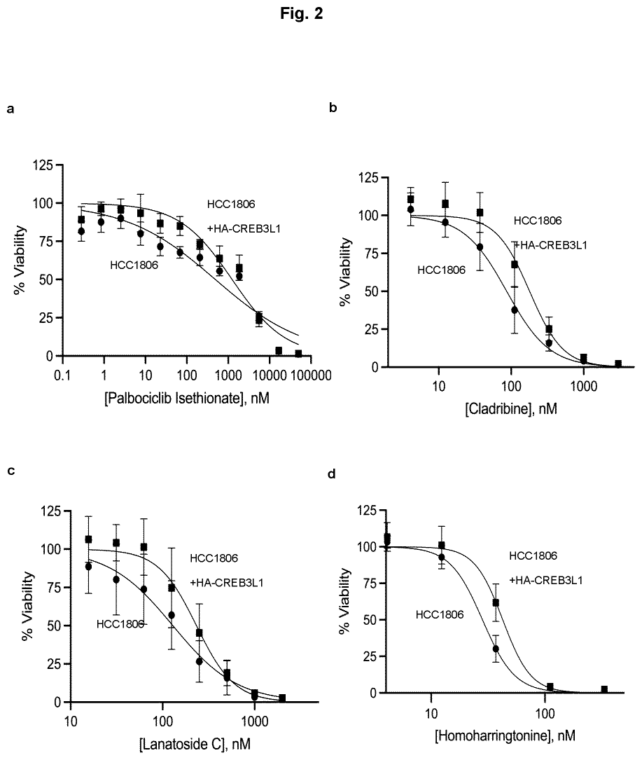

Drug Titration experiments were carried out on these 27 drugs to determine their EC values (i.e. the concentration of a drug necessary to reach half of the maximum response) in order to compare the efficacy of each drug (FIG. 1). The majority of drugs (20 drugs) were similarly cytotoxic to both cell lines or showed more cytotoxicity towards the HCC1806+HACREB3L1 cells. There were 4 drugs that showed a preference for killing the more metastatic CREB3L1-deficient HCC1806 TNBC cells, palbociclib isethionate, cladribine, homoharringtonine and lanatoside C (FIG. 2a-d).

### Evaluation of Promising Drugs in Multiple Breast Cell Lines

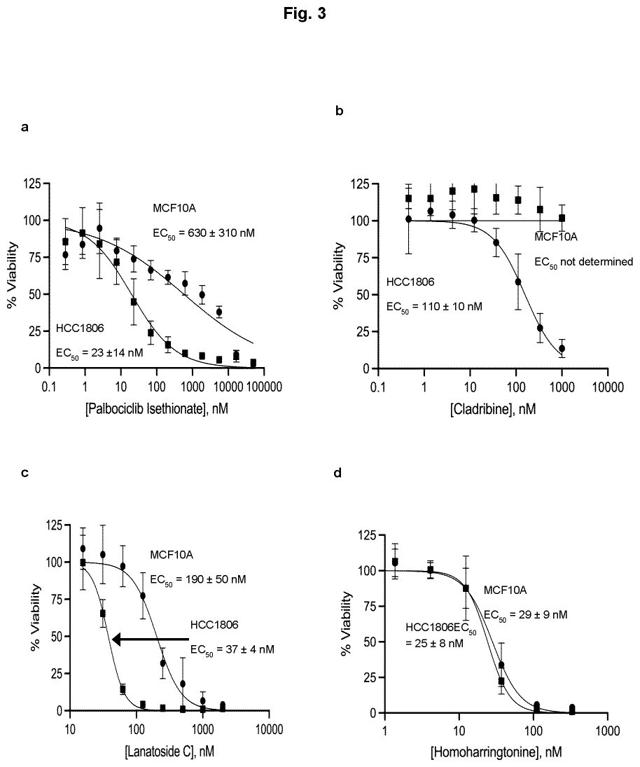

To assess the cytotoxicity towards a non-tumorigenic breast cell line MCF10A, similar cytotoxicity determinations as done with the HCC1806 TNBC cells were carried out (FIG. 3). MCF10A cells were very sensitive to palbociclib isethionate and lanatoside C, with much lower EC values than the TNBC HCC1806 cells (FIG. 3a, c). Since palbociclib is currently used clinically, this observed cytotoxicity may not preclude its use. Little or no cytotoxicity was observed when MCF10A cells were treated with cladribine (FIG. 3b), suggesting that it may not have adverse effects towards normal tissue. The cytotoxicity profile of homoharringtonine was similar for both non-tumorigenic MCF10A breast cells and TNBC HCC1806 cells (FIG. 3d).

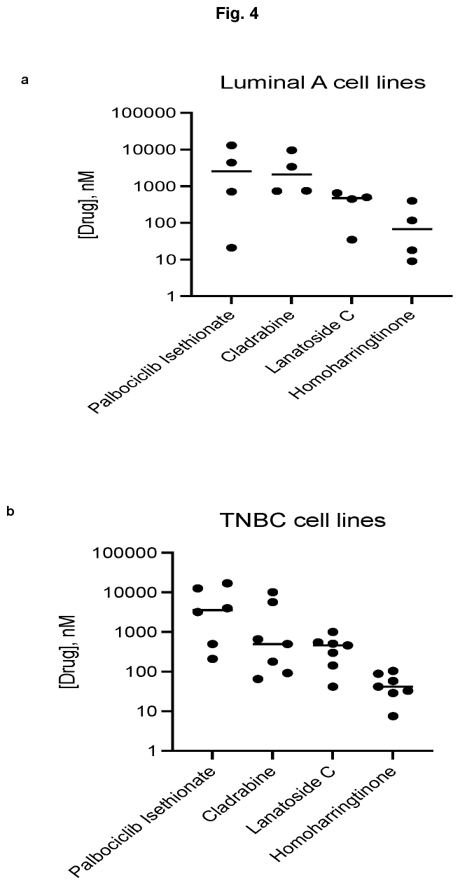

The 4 promising drugs which showed more cytotoxic effects towards the more metastatic CREB3L1-deficient HCC1806 TNBC cells were of particular interest since these are typically the most challenging types of cancer cells to treat. To assess the utility of these drugs across additional CREB3L1-deficient breast cancer cells, the cytotoxicity of palbociclib isethionate, cladribine, lanatoside C and homoharringtonine across a panel of CREB3L1-deficient TNBC cell lines, including BT549, HCC1395, HCC38, Hs578T, MDA-MB-157 and MDA-MB-436 was evaluated (Table 1). Since luminal A breast cancers that are CREB3L1-deficient also show a poor prognosis as compared to those expressing CREB3L1 [11], this analysis was extended to determine the drug sensitivity in several luminal A breast cancer cell lines, some of which were also tested with re-expressed CREB3L1 (Table 1). HCC1428 luminal A breast cancer cells showed increased drug sensitivity to both palbociclib isethionate and cladribine as compared to the corresponding CREB3L1-expressing cells, however the opposite was true for lanatoside C and homoharringtonine. The TNBC HCC1806 cells exhibited a CREB3L1-dependent drug sensitivity for all four drugs. Thus, drug sensitivity may be impacted differently by CREB3L1 expression in different breast cancer cell lines, or in different breast cancer subtypes. Overall, a range of drug sensitivities were observed, but both luminal A and TNBC cells were most sensitive to homoharringtonine (FIG. 4).

TABLE 1
EC₅₀ values across multiple CREB3L1-deficient TNBC and luminal A breast cancer
cell lines. Cell viability was determined over a range of drug concentrations
after 4 days of drug treatment and EC50 values were determined.

| | | Palbociclib | | | |
|---|---|---|---|---|---|
| | | Isethionate | | Cladribine | |
| | | | Parental + | | Parental + |
| | | | HA- | | HA- |
| | | Parental | CREB3 | Parental | CREB3 |
| Drug | | EC50, | L1 EC₅₀, | EC₅₀, | L1 EC₅₀, |
| Name | Cell Line | nM (n¹) | nM (n¹) | nM (n¹) | nM (n¹) |
| TNBC | BT549 | 12600 ± 4800 (9) | | 178 ± 84 (9) | |
| | HCC1395 | 4000 ± 2000 (6) | | 10100 ± 1200 (6) | |
| | HCC1806 | 500 ± 130 (9) | 1300 ± 490 (9) | 92 ± 44 (9) | 182 ± 60 (9) |
| | HCC38 | 210 ± 70 (9) | | 65 ± 56 (9) | |
| | Hs578T | 3200 ± 1400 (4) | | 5700 ± 1200 (4) | |
| | MDA-MB- | ND | | 5000 ± 4300 (4) | |
| | 157 | | | | |
| | MDA-MB- | 17000 ± 14000 (4) | | 660 ± 180 (4) | |
| | 436 | | | | |
| Luminal | HCC1428 | 21 ± 15 (4) | 160 ± 250 (4) | 740 ± 330 (4) | 2200 ± 1700 (4) |
| A (ER+) | | | | | |
| | MCF7 | 4400 ± 3500 (4) | | 3400 ± 2100 (4) | |
| | T47D | 13000 ± 9800 (4) | 7200 ± 9200 (4) | 9600 ± 1800 (6) | 1800 ± 1300 (6) |
| | ZR-75-1 | 710 ± 350 (8) | | 750 ± 350 (9) | |

| | | | Homoharring- | |
|---|---|---|---|---|
| | Lanatoside C | | tonine | |
| | | Parental + | | Parental + |
| | | HA- | | HA- |
| | Parental | CREB3 | Parental | CREB3L1 |
| Drug | EC₅₀, | L1 EC₅₀, | EC₅₀, | EC50, nM |
| Name | nM (n¹) | nM (N1) | nM (n¹) | (n¹) |
| TNBC | 42 ± 12 (9) | | 89 ± 10 (9) | |
| | 510 ± 300 (6) | | 104 ± 31 (6) | |
| | 143 ± 85 (9) | 233 ± 100 (9) | 29 ± 4 (9) | 42 ± 7 (9) |
| | 300 ± 100 (9) | | 58 ± 10 (9) | |
| | 1000 ± 800 (4) | | 42 ± 24 (4) | |
| | 550 ± 240 (4) | | 7.5 ± 1.2 (4) | |
| | 460 ± 250 (4) | | 33 ± 36 (4) | |
| Luminal | 35 ± 8 (4) | 32 ± 8 (4) | 9 ± 3 (4) | 10 ± 3 (4) |
| A (ER+) | | | | |
| | 450 ± 490 (4) | | 18 ± 9 (4) | |
| | 500 ± 50 (4) | 89 ± 34 (4) | 400 ± 180 (6) | 88 ± 55 (6) |
| | 650 ± 510 (9) | | 117 ± 35 (9) | |

¹n, number of replicate measurements

Combining Promising Drugs with Either Paclitaxel or Doxorubicin

Since new drug treatments are potentially to be offered in combination with existing therapies, possible synergistic effects between some of the currently used chemotherapy agents and the four most promising drugs identified was evaluated. Cytotoxic chemotherapy agents which can be used for TNBC patients include the anthracycline doxorubicin and the microtubule stabilizing drug paclitaxel.

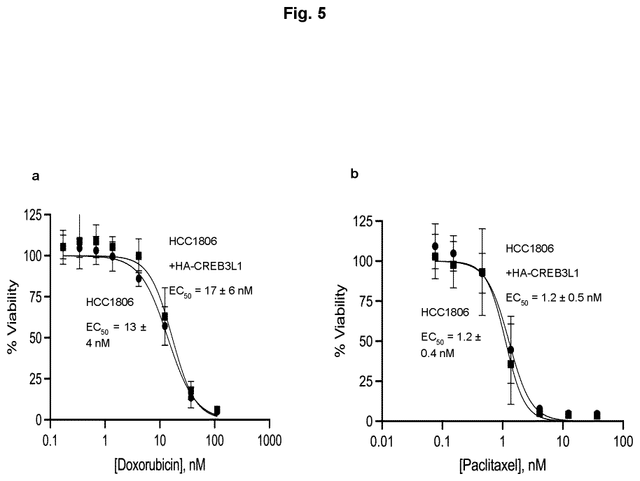

Doxorubicin and paclitaxel and were each tested separately, in pairwise combination drug titration experiments with each of palbociclib isethionate, cladribine, homoharringtonine and lanatoside C. Drug titration experiments were initially carried out for paclitaxel and doxorubicin individually in HCC1806+HA-CREB3L1 cells, to determine EC values for each drug when used alone (FIG. 5). EC₅₀ values for doxorubicin were very similar for the two cell lines with 13±4 nM for HCC1806 and 17±6 nM for HCC1806+HA-CREB3L1 (FIG. 5). EC₅₀ values for paclitaxel were also very similar for the two cell lines with 1.2±0.5 nM for HCC1806 and 1.2±0.4 nM for HCC1806+HA-CREB3L1 (FIG. 5).

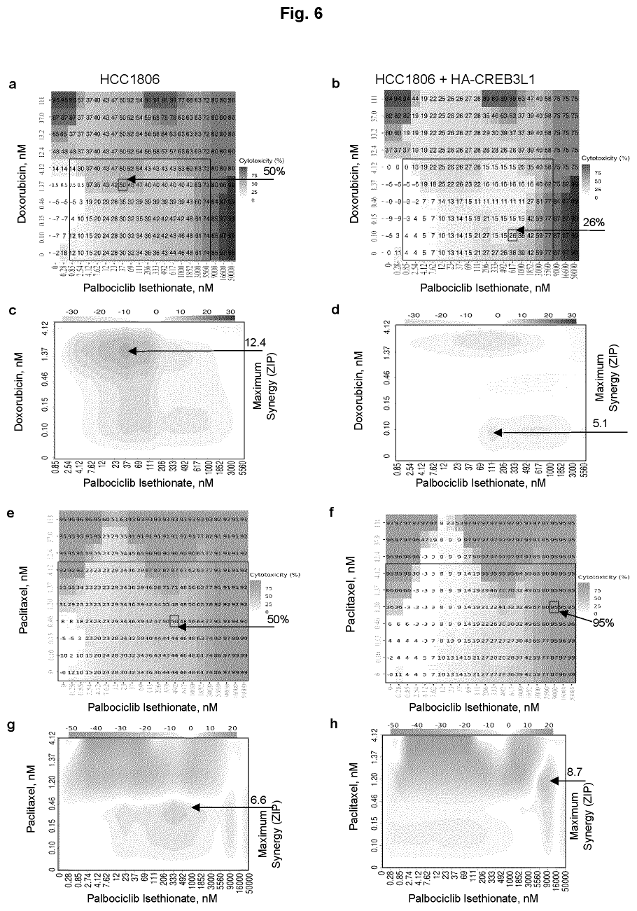

A cross-design combination experiment was used as detailed in the methods where drug 1 is tested over a range of concentrations and drug 2 is maintained at its EC₅₀ value. A second set of experiments then tested drug 2 over a range of concentrations and drug 1 is maintained at its EC₅₀ value. Cells were treated for four days and cell viability assessed as before. The SynergyFinder® package was used to determine the possible additive, synergistic, or antagonistic effect of the combined drugs [17]. This recently developed synergy model improves upon previous scoring models including Highest simple agent, Lowe additivity and Bliss models [18]. The SynergyFinder package characterizes a synergy landscape of drug interaction by calculating a Zero Interaction Potency (ZIP) score defining delta scores for every input data point and interpolating untested data points in between [17]. The ZIP defined delta score is the additional response (% Inhibition) observed beyond the expected effect (as determined by the ZIP model) for the given concentrations of two drugs [18]. For example, a positive ZIP defined delta score of 10 (red in the synergy landscape) indicates that the response observed had a 10% higher inhibition than would be expected if the combined drug effect was non-interactive or additive, with values >10 indicating significant synergy (FIGS. 6-9; Table 2).

TABLE 2
Synergy of Drug Combinations

| | | HCC1806 | | HCC1806 + HA-CREB3L1 | |
|---|---|---|---|---|---|
| Drug 1 | Drug 2 | ZIP² | % | ZIP² | % |
| (EC₅₀¹) | (EC₅₀1) | (drug1; drug2) | cytotoxicity | (drug1; drug2) | cytotoxicity |
| Palbociclib | Doxorubicin | 12.4 | 50 | 5.1 | 26 |
| Isethionate | (13.2 nM) | (37 nM; 1.37 nM) | | (111 nM; 0.08 nM) | |
| (500 nM) | | | | | |
| Palbociclib | Paclitaxel | 6.6 | 50 | 8.7 | 95 |
| Isethionate | (1.2 nM) | (492 nM; 0.46 nM) | | (9000 mM; 1.2 nM) | |
| (500 nM) | | | | | |
| Cladribine | Doxorubicin | 16.2 | 21 | 18.8 | 24 |
| (92 nM) | (13.2 nM) | (12 nM; 1.37 nM) | | (37 nM; 4.12 nM) | |
| Cladribine | Paclitaxel | 9.3 | 15 | 24 | 41 |
| (92 nM) | (1.2 nM) | (12 nM; 0.15 nM) | | (79 nM; 0.46 nM) | |
| Lanatoside C | Doxorubicin | 1.3 | 55 | 9.1 | 17 |
| (143 nM) | (13.2 nM) | (31 nM; 111 nM) | | (31 nM; 4.12 nM) | |
| Lanatoside C | Paclitaxel | 2.8 | 96 | 12 | 53 |
| (143 nM) | (1.2 nM) | (16 nM; 111 nM) | | (151 nM; 0.46 nM) | |
| Homoharring | Doxorubicin | 15.7 | 21 | 12.7 | 14 |
| tonine | (13.2 nM) | (12 nM; 0.46 nM) | | (12 nM; 1.37 nM) | |
| (29 nM) | | | | | |
| Homoharring | Paclitaxel | 51 | 76 | 59 | 83 |
| tonine | (1.2 nM) | (12 nM; 0.15 nM) | | (12 nM; 0.15 nM) | |
| (29 nM) | | | | | |

¹EC₅₀ as single agent in HCC1806 cells.
²ZIP, zero interaction potency; scores >10 indicate synergy.

Some synergy was observed using palbociclib isethionate and doxorubicin for the CREB3L1-deficient HCC1806 cells, with a maximum ZIP synergy score of 12.4 giving rise to 50% cytotoxicity (FIG. 6a, c; Table 2). Synergy was not observed for these two drugs for the HCC1806+HA-CREB3L1 cells, suggesting that the synergy is CREB3L1-deficiency dependent (FIG. 6b, d; Table 2). A strong antagonistic effect was observed at 111 nM paclitaxel and 12, 23, and 37 nM palbociclib, suggesting they are less cytotoxic when used at these concentrations (FIG. 6 4e-f). No positive synergy was observed for the palbociclib and paclitaxel drug combination (FIG. 6e-h; Table 2).

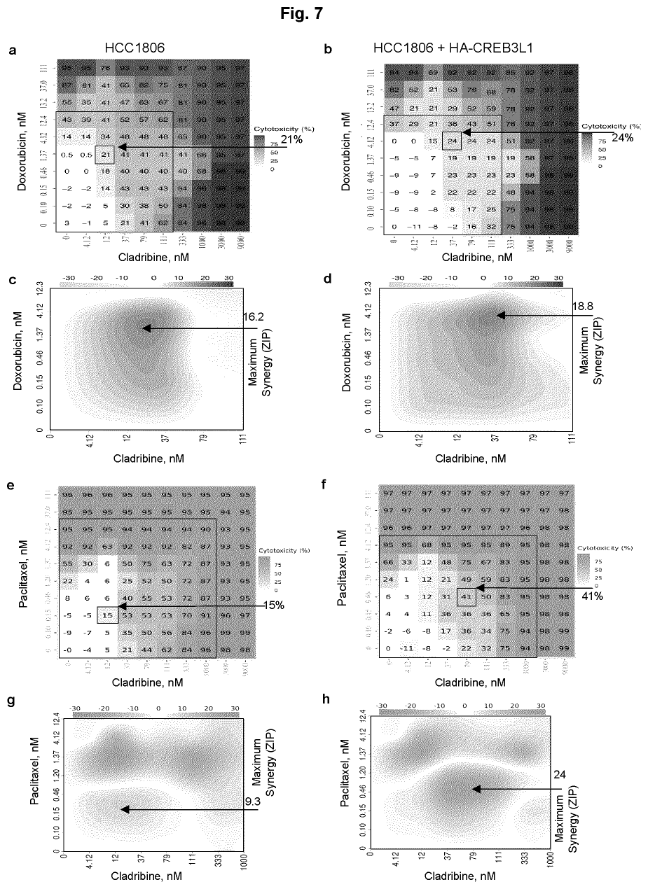

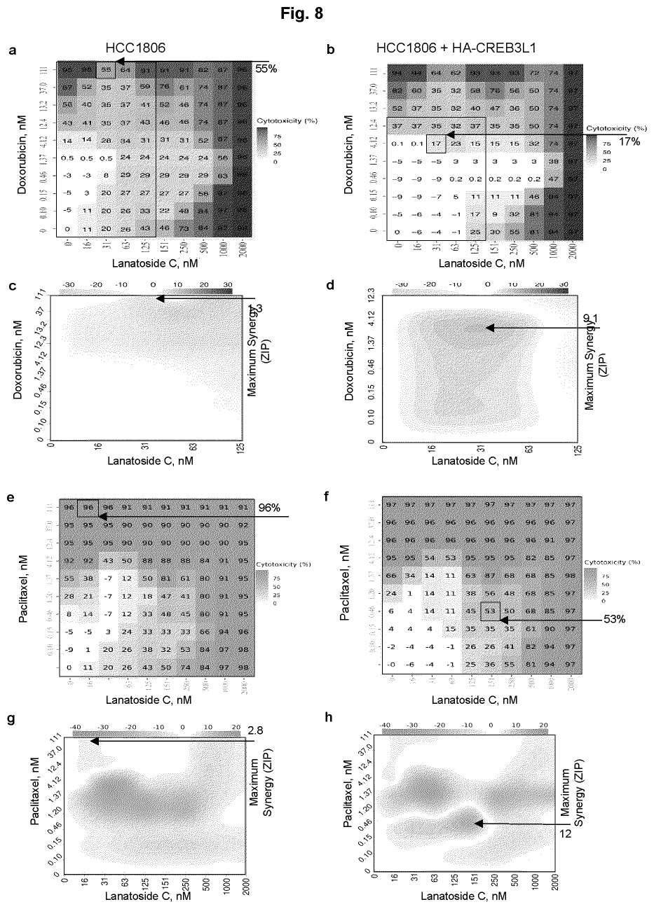

Cladribine exhibited synergistic cytotoxicity when combined with doxorubicin in both cell lines, suggesting CREB3L1-independent synergy, with ZIP scores of 16.2 and 18.8, with corresponding cytotoxicity of 21% and 24% for HCC1806 and HCC1806+HA-CREB3L1 cells, respectively (FIG. 7a-d; Table 2). Cladribine was also selectively synergistic with paclitaxel but only for HA-CREB3L1 expressing cells and not the HCC1806 cells (FIG. 7e-h; Table 2). In contrast, lanatoside C did not display any synergy with doxorubicin but was synergistic towards HCC1806+HA-CREB3L1 cells when combined with paclitaxel (FIG. 8, Table 2).

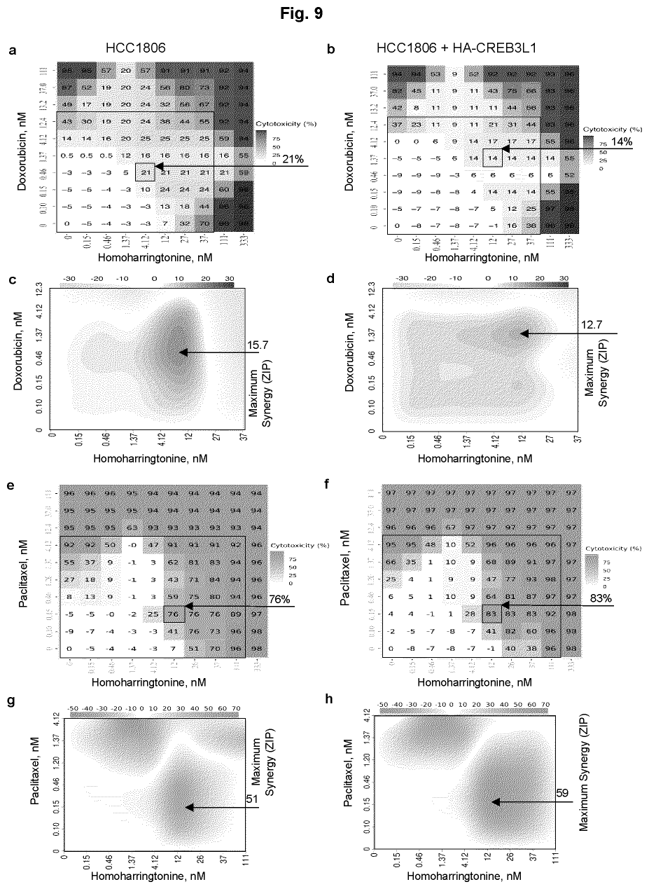

The strongest synergy was exhibited by homoharringtonine, which showed similar effects in HCC1806+HA-CREB3L1 cells, suggesting CREB3L1-independent effects (FIG. 9, Table 2). When paired with doxorubicin, the ZIP synergy scores for homoharringtonine were 15.7 and 12.7, however this resulted in fairly low cell cytotoxicity of 21% and 14%. When homoharringtonine and paclitaxel were used in combination, they provided a very high ZIP synergy score of 51 and 59, and a corresponding high cytotoxicity of 76% and 83% (FIG. 9; Table 2). The most synergistic drug combination was achieved using 12 nM homoharringtonine and 0.15 nM paclitaxel, resulting in 76-83% cytotoxicity (Table 2). These drug doses were considerably lower than the EC values of individual drugs when used alone, with homoharringtonine (EC₅₀₌₂₉ nM) and paclitaxel (EC_{50=1.2} nM). These promising results suggest that homoharringtonine and paclitaxel combination treatment could be very effective in treating TNBCs using relatively low concentrations such that toxic side-effects would be minimized.

### Discussion

Homoharringtonine blocks protein synthesis by transiently binding to ribosomes and inhibiting protein translation [19]. This results in a selective loss of proteins with short half-lives, such as those regulating cell proliferation and cell survival [20, 21]. Homoharringtonine has been shown to rapidly reduce the expression of several anti-apoptotic proteins, including MCL-1, BCL-2 and surviving in several TNBC cell lines [21]. Thus, while not being bound by theory, the cytotoxicity observed is likely due to the loss of MCL-1 expression causing apoptosis.

The results herein are consistent with a genome-wide siRNA lethality screen that identified a large overrepresentation of proteasome genes as a specific dependency in basal-like TNBCs and this was shown to be mediated by NOXA and MCL-1 [22]. Proteasome inhibition was further shown to reduce the growth of basal-like TNBC tumors in mouse xenograft studies [22]. Similar results were seen upon treatment of MDA-MB-231 TNBC xenografts with homoharringtonine with reduced tumor growth without general toxicity for the mice [21].

Overexpression of MCL-1 in several TNBC cell lines has been shown to increase resistance to the chemotherapy agent's doxorubicin and docetaxel [23]. The MCL-1 inhibitor S63845 displayed synergistic activity with docetaxel in TNBC, suggesting that blocking MCL-1 function may sensitize cells to cytotoxic chemotherapeutic agents by reducing this pro-survival activity [23]. MCL-1 levels can be upregulated even higher in breast tumor samples from patients treated with a variety of neoadjuvant chemotherapy agents including doxorubicin, cyclophosphamide and fluorouracil [24]. While not being bound by theory, these results suggest that increased MCL-1 expression protects TNBCs from chemotherapy-induced apoptosis [24]. The results herein suggest that homoharringtonine may similarly reduce the levels of MCL-1 and sensitize TNBC cells to the cytotoxic agent paclitaxel. The strong synergy observed for the homoharringtonine-paclitaxel combination treatment suggests it may be both effective and have reduced toxic side-effects, an ideal combination for patients with TNBC.

The large scale drug screen identified a number of compounds that are cytotoxic towards TNBC cells. The most effective of these was the protein translation inhibitor homoharringtonine, which has been shown to be highly synergistic when combined with paclitaxel. While not being bound by theory, the results herein suggest that TNBC cells may be particularly sensitive to combination homoharringtonine-paclitaxel treatment and that effective TNBC cell killing can be achieved using considerably lower drug concentrations for each, reducing their toxic side-effects yet maintaining their anti-cancer efficacy.

### Example 2

### Homoharringtonine and Paclitaxel Combination Therapy are Effective Across a Panel of Triple Negative Breast Cancer (TNBC) Cell Lines.

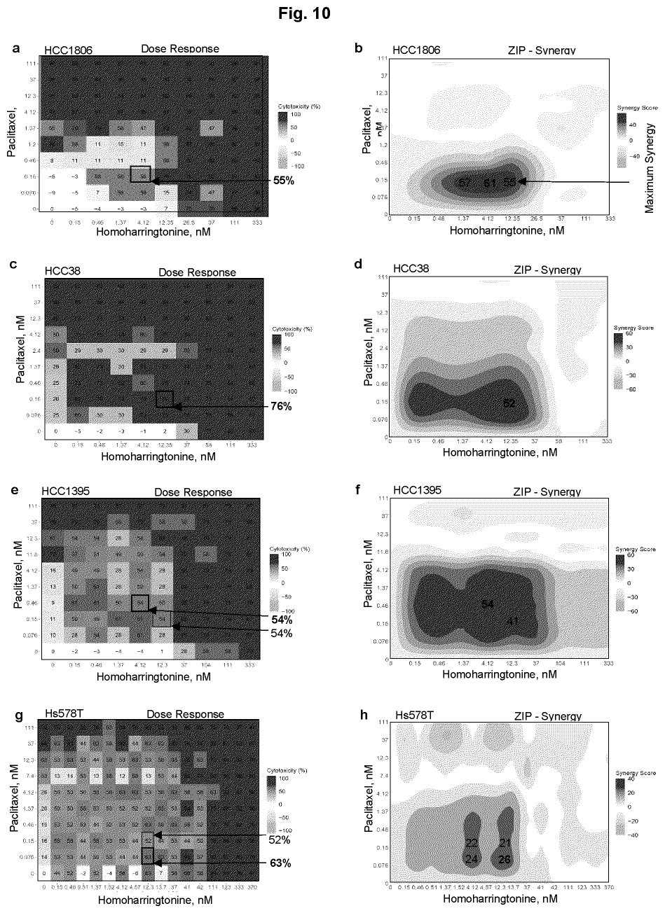

As the combination homoharringtonine (HHT) and paclitaxel (PTX) treatment showed strong synergy in the TNBC cell line HCC1806 in Example 1, the synergy measurements were repeated in HCC1806 cells and the analysis further expanded to three additional TNBC cell lines, HCC38, HCC1395 and Hs578T. First, EC₅₀ values were determined for each of drug alone (Table 3). A synergy analysis was then carried out as previously detailed in Example 1). The combination of HHT and PTX showed strong synergy in all four TNBC cell lines evaluated (ZIP synergy scores 26-61), with good cytotoxicity (54-76%) (FIG. 10; Table 3). The drug concentrations showing maximum synergy were similar across the TNBC cell lines: HHT (4-12 nM) and PTX (0.076-0.15 nM). At HHT (12 nM) and PTX (0.15 nM), strong synergy was observed in the four TNBC cell lines, suggesting a consistent and robust response to this new combination therapy.

TABLE 3
Synergy of drug combinations across additional TNBC cell lines.

| | | | | | | | Overall optimal | |
|---|---|---|---|---|---|---|---|---|
| | | | | | | | [HHT] 12 nM; | |
| | EC50¹, each drug | | Max ZIP² Synergy Score | | | | [PTX] 0.15 nM | |
| | alone | | | | ZIP² | % | ZIP² | % |
| Cell line | [HHT], nM | [PTX], nM | [HHT], nM | [PTX], nM | score | cytotoxicity | score | cytotoxicity |
| HCC1806 | 29 | 1.2 | 4 | 0.15 | 61 | 55 | 51-55 | 76 |
| HCC38 | 58 | 2.4 | 12 | 0.15 | 52 | 76 | 52 | 76 |
| HCC1395 | 104 | 12 | 4 | 0.46 | 53 | 54 | 41 | 54 |
| Hs578T | 42 | 7.4 | 12 | 0.076 | 26 | 63 | 21 | 52 |

¹EC₅₀ as single agent.
²ZIP, zero-interaction potency; scores >10 indicate synergy.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Full Citations for Documents Referred to in the Specification

A number of publications are cited herein. Full citations for these references are provided below. Each of these references is incorporated herein by reference in its entirety into the present disclosure, to the same extent as if each individual reference was specifically and individually indicated to be incorporated by reference.
1. Government of Canada-Public Health Agency of Canada, website: http://www.phac-aspc.gc.ca/cd-mc/cancer/breast_cancer-cancer_du_sein-eng.php; Breast Cancer, 21/04/2021.
2. American Cancer Society: http://www.cancer.org/cancer/breastcancer/detailedguide/breast-cancer-survival-by stage, Breast Cancer, 21/04/2021
3. Canadian Cancer Society website: https://cancer.ca/en/cancer-information/cancertypes/breast/statistics?_ga=2.263777804.1429687422.1641410797-1677001365.1619734808, Breast Cancer Statistics, 21/04/2021
4. Eroles P, Bosch A, Perez-Fidalgo J A, Lluch A: Molecular biology in breast cancer: intrinsic subtypes and signaling pathways. Cancer treatment reviews 2012, 38 (6): 698-707.
5. Rampurwala M M, Rocque G B, Burkard M E: Update on adjuvant chemotherapy for early breast cancer. Breast Cancer (Auckl) 2014, 8:125-133.
6. Chan C P, Kok K H, Jin D Y: CREB3 subfamily transcription factors are not created equal: Recent insights from global analyses and animal models. Cell Biosci 2011, 1 (1): 6.
7. Ye J: Transcription factors activated through RIP (regulated intramembrane proteolysis) and RAT (regulated alternative translocation). J Biol Chem 2020, 295 (30): 10271-10280.
8. Sampieri L, Di Giusto P, Alvarez C: CREB3 Transcription Factors: ER-Golgi Stress Transducers as Hubs for Cellular Homeostasis. Front Cell Dev Biol 2019, 7:123.
9. Saito A: Physiological functions of endoplasmic reticulum stress transducer OASIS in central nervous system. Anatomical science international 2014, 89 (1): 11-20.
10. Mellor P, Deibert L, Calvert B, Bonham K, Carlsen S A, Anderson D H: CREB3L1 is a metastasis suppressor that represses expression of genes regulating metastasis, invasion and angiogenesis. Mol Cell Biol 2013, 33 (24): 4985-4995.
11. Ward A K, Mellor P, Smith S E, Kendall S, Just N A, Vizeacoumar F S, Sarker S, Phillips Z, Alvi R, Saxena A et al: Epigenetic silencing of CREB3L1 by DNA methylation is associated with high-grade metastatic breast cancers with poor prognosis and is prevalent in triple negative breast cancers. Breast Cancer Research 2016, 18 (1): 12.
12. Rose M, Schubert C, Dierichs L, Gaisa N T, Heer M, Heidenreich A, Knuchel R, Dahl E: OASIS/CREB3L1 is epigenetically silenced in human bladder cancer facilitating tumor cell spreading and migration in vitro. Epigenetics: official journal of the DNA Methylation Society 2014, 9 (12): 1626-1640.
13. Denard B, Jiang S, Peng Y, Ye J: CREB3L1 as a potential biomarker predicting response of triple negative breast cancer to doxorubicin-based chemotherapy. BMC cancer 2018, 18 (1): 813.
14. Mellor P, Kendall S, Smith S, Saxena A, Anderson D H: Reduced CREB3L1 expression in triple negative and luminal A breast cancer cells contributes to enhanced cell migration, anchorage-independent growth and metastasis. PLOS One 2022, Under Revision.
15. Vizeacoumar F J, Arnold R, Vizeacoumar F S, Chandrashekhar M, Buzina A, Young J T, Kwan J H, Sayad A, Mero P, Lawo S et al: A negative genetic interaction map in isogenic cancer cell lines reveals cancer cell vulnerabilities. Molecular systems biology 2013, 9:696.
16. Malyutina A, Majumder M M, Wang W, Pessia A, Heckman C A, Tang J: Drug combination sensitivity scoring facilitates the discovery of synergistic and efficacious drug combinations in cancer. PLOS Comput Biol 2019, 15 (5): e1006752.
17. He L, Kulesskiy E, Saarela J, Turunen L, Wennerberg K, Aittokallio T, Tang J: Methods for High-throughput Drug Combination Screening and Synergy Scoring. Methods in molecular biology 2018, 1711:351-398.
18. Yadav B, Wennerberg K, Aittokallio T, Tang J: Searching for Drug Synergy in Complex Dose-Response Landscapes Using an Interaction Potency Model. Comput Struct Biotechnol J 2015, 13:504-513.
19. Gurel G, Blaha G, Moore P B, Steitz T A: U2504 determines the species specificity of the A-site cleft antibiotics: the structures of tiamulin, homoharringtonine, and bruceantin bound to the ribosome. J Mol Biol 2009, 389 (1): 146-156.
20. Lu S, Wang J: Homoharringtonine and omacetaxine for myeloid hematological malignancies. J Hematol Oncol 2014, 7:2.
21. Yakhni M, Briat A, El Guerrab A, Furtado L, Kwiatkowski F, Miot-Noirault E, Cachin F, Penault-Llorca F, Radosevic-Robin N: Homoharringtonine, an approved anti-leukemia drug, suppresses triple negative breast cancer growth through a rapid reduction of anti-apoptotic protein abundance. American journal of cancer research 2019, 9 (5): 1043-1060.
22. Petrocca F, Altschuler G, Tan S M, Mendillo M L, Yan H, Jerry D J, Kung A L, Hide W, Ince T A, Lieberman J: A genome-wide siRNA screen identifies proteasome addiction as a vulnerability of basal-like triple-negative breast cancer cells. Cancer Cell 2013, 24 (2): 182-196.
23. Merino D, Whittle J R, Vaillant F, Serrano A, Gong J N, Giner G, Maragno A L, Chanrion M, Schneider E, Pal B et al: Synergistic action of the MCL-1 inhibitor S63845 with current therapies in preclinical models of triple-negative and HER2-amplified breast cancer. Science translational medicine 2017, 9 (401).
24. Balko J M, Giltnane J M, Wang K, Schwarz L J, Young C D, Cook R S, Owens P, Sanders M E, Kuba M G, Sanchez V et al: Molecular profiling of the residual disease of triple-negative breast cancers after neoadjuvant chemotherapy identifies actionable therapeutic targets. Cancer Discov 2014, 4 (2): 232-245

1. A method of treating breast cancer comprising administering, to a subject in need thereof, an effective amount of paclitaxel, or a pharmaceutically acceptable salt, prodrug and/or solvate thereof, in combination with an effective amount of homoharringtonine or a pharmaceutically acceptable salt, prodrug and/or solvate thereof.

2. A method of improving the efficacy of homoharringtonine for treating breast cancer comprising administering, to a subject in need thereof, an effective amount of paclitaxel, or a pharmaceutically acceptable salt, prodrug and/or solvate thereof, in combination with an effective amount of homoharringtonine or a pharmaceutically acceptable salt, prodrug and/or solvate thereof.

3. A method of preventing or reducing breast cancer metastasis comprising administering, to a subject in need thereof, an effective amount of homoharringtonine, or a pharmaceutically acceptable salt, prodrug and/or solvate thereof, in combination with an effective amount of paclitaxel or a pharmaceutically acceptable salt, prodrug and/or solvate thereof.

4. The method of any one of claims 1 to 3, wherein the breast cancer is selected from triple negative breast cancer, basal-like breast cancer, luminal A breast cancer, luminal B breast cancer, BRCA1 breast cancer, BRCA2 breast cancer, HER2+ breast cancer and combinations thereof.

5. The method of claim 4, wherein the breast cancer is selected from triple negative breast cancer, basal-like breast cancer and Luminal A breast cancer and combinations thereof.

6. The method of claim 4, wherein the breast cancer is selected from triple negative breast cancer or basal-like breast cancer and combinations thereof.

7. The method of claim 4, wherein the breast cancer is triple negative breast cancer.

8. A method for treating breast cancer, for improving the efficacy of homoharringtonine for treating breast cancer, and for preventing or reducing breast cancer metastasis comprising administering, to a subject in need thereof, a therapeutically effective amount homoharringtonine, or a pharmaceutically acceptable salt, prodrug and/or solvate thereof, in combination with an therapeutically effective amount of paclitaxel or a pharmaceutically acceptable salt, prodrug and/or solvate thereof further in combination with another known agent useful for treatment of breast cancer.

9. The method of claim 8, wherein the additional breast cancer treatment is selected from one or more of radiotherapy, chemotherapy, targeted therapies such as antibody therapies, small molecule therapies such as tyrosine-kinase inhibitors therapies, glutaminase inhibitors (GLS1 inhibitors), immunotherapy, hormonal therapy, surgical intervention, radiation therapy and anti-angiogenic therapies.

10. The method of any one of claims 1 to 9, wherein the effective amount is a synergistically effective amount.

11. The method of any one of claims 1 to 10, wherein the homoharringtonine, or a salt, prodrug and/or solvate thereof and the paclitaxel, or a salt, prodrug and/or solvate thereof, are administered at dosages that are equal to or less than a dosage of the homoharringtonine, or a salt, prodrug and/or solvate thereof and the paclitaxel, or a salt, prodrug and/or solvate thereof when each is used alone.

12. The method of any one of claims 1 to 11, wherein the homoharringtonine, or a salt, prodrug and/or solvate thereof is administered subcutaneously about every 12 hours for about 2 weeks and the paclitaxel, or a salt, prodrug and/or solvate thereof is administered about three times per week every three weeks.

13. The method of claim 11, wherein the homoharringtonine, or a salt, prodrug and/or solvate thereof, is administered subcutaneously at dosages of about 1.25 mg/m² to about 1.00 mg/m².

14. The method of claim 11, wherein the paclitaxel or a salt, prodrug and/or solvate thereof, is administered orally at dosages of about 175 mg/m² to about 205 mg/m².

15. A pharmaceutical composition comprising homoharringtonine or a pharmaceutically acceptable salt, prodrug and/or solvate thereof and paclitaxel or a pharmaceutically acceptable salt, prodrug and/or solvate thereof wherein the homoharringtonine and paclitaxel are present in amounts that are effective to treat breast cancer, for improving the efficacy of homoharringtonine for treating breast cancer or for preventing or reducing breast cancer metastasis.

16. The pharmaceutical composition of claim 15, wherein the amounts that are effective are synergistic effective amounts.

17. A kit for the treatment breast cancer, the kit comprising paclitaxel or a pharmaceutically acceptable salt, prodrug and/or solvate thereof and homoharringtonine or a pharmaceutically acceptable salt, prodrug and/or solvate thereof and optionally instructions for administration of paclitaxel or a pharmaceutically acceptable salt, prodrug and/or solvate thereof and homoharringtonine, or a pharmaceutically acceptable salt and/or solvate thereof, to a subject in need thereof.

18. A kit for the treatment of a breast cancer, the kit comprising: homoharringtonine or a pharmaceutically acceptable salt and/or solvate thereof; and instructions for administration of homoharringtonine, or a pharmaceutically acceptable salt and/or solvate thereof, to a subject being administered paclitaxel for treating breast cancer.

19. A kit for the treatment of a breast cancer, the kit comprising: paclitaxel or a pharmaceutically acceptable salt and/or solvate thereof; and instructions for administration of paclitaxel, or a pharmaceutically acceptable salt and/or solvate thereof, to a subject being administered homoharringtonine for treating breast cancer.

20. A kit for improving the efficacy of homoharringtonine for the treatment of breast cancer, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

21. A kit for improving the efficacy of homoharringtonine for the treatment breast cancer, the kit comprising homoharringtonine, or a pharmaceutically acceptable salt and/or solvate thereof; and instructions for administration of homoharringtonine, or a pharmaceutically acceptable salt and/or solvate thereof, to a subject being administered paclitaxel for the treatment of breast cancer.

22. A kit for preventing or reducing breast cancer metastasis, the kit comprising homoharringtonine or a pharmaceutically acceptable salt, prodrug and/or solvate thereof and paclitaxel or a pharmaceutically acceptable salt, prodrug and/or solvate thereof; and optionally instructions for administration of the homoharringtonine or a pharmaceutically acceptable salt, prodrug and/or solvate thereof and paclitaxel or a pharmaceutically acceptable salt, prodrug and/or solvate thereof, to a subject in need thereof.

23. The kit of any one of claims 17 to 22, wherein the breast cancer is selected from triple negative breast cancer, basal-like breast cancer, luminal A breast cancer, luminal B breast cancer, BRCA1 breast cancer, BRCA2 breast cancer, HER2+ breast cancer and combinations thereof.

24. The kit of claim 23, wherein the breast cancer is selected from triple negative breast cancer, basal-like breast cancer and luminal A breast cancer and combinations thereof.

25. The kit of claim 23, wherein the breast cancer is triple negative breast cancer.
